(19) United States
(12) Patent Application Publication
(10) Pub. No.: US 2017/0226145 A1
(43) Pub. Date: Aug. 10, 2017

(54) METHODS OF EXTRACTION AND PURIFICATION FROM STEVIA REBAUDIANA OF COMPOSITIONS WITH ENHANCED REBAUDIOSIDE-M CONTENT, USES OF SAID COMPOSITION AND NATURAL SWEETENER COMPOSITIONS WITH SAID COMPOSITION

(71) Applicant: GLG LIFE TECH CORPORATION, Richmond (CA)

(72) Inventors: Yong Luke Zhang, Richmond (CA); Cunbiao Kevin Li, Richmond (CA)

(73) Assignee: GLG LIFE TECH CORPORATION, Richmond, BC (CA)

(21) Appl. No.: 15/503,376

(22) PCT Filed: Aug. 12, 2015
(86) PCT No.: PCT/CA2015/000462

§ 371 Date: Feb. 10, 2017

(30) Foreign Application Priority Data
Aug. 12, 2014 (CN) 201410393477.0

(51) Int. Cl. C07H 15/256; C07H 1/08; A23L 33/105; A23L 2/60; A23L 27/30; A61K 47/26; B01D 15/36

(52) U.S. Cl. CPC C07H 15/256; A61K 47/26; C07H 1/08; B01D 15/361; A23L 2/60; A23L 27/36; A23L 33/105; A23V 2002/00

(57) ABSTRACT

A method for purifying Reb M includes preparing a crude mother liquor/Stevia rebaudiana primary extract, passing a solution of this primary extract through a multi-column system including a plurality of columns, in series, packed with a porous adsorbent resin to provide at least one column having adsorbed Reb M and eluting fractions with Reb M content from a final/last column in the series to provide an eluted solution with Reb M content.

### FIELD OF THE INVENTION

The present invention relates generally to methods of extracting natural products from plants, in particular from Stevia rebaudiana.

### BACKGROUND

In the food and beverage industry, there is a general preference for the consumption of sweet foods, and manufacturers and consumers commonly add sugar in the form of sucrose (table sugar), fructose or glucose to beverages, food, etc. to increase the sweet quality of the beverage or food item. Although most consumers enjoy the taste of sugar, sucrose, fructose and glucose are high calorie sweeteners. Many alternatives to these high calorie sweeteners are artificial sweeteners or sugar substitutes, which can be added as an ingredient in various food items.

Common artificial sweeteners include saccharin, aspartame, and sucralose. Unfortunately, these artificial sweeteners have been associated with negative side effects. Therefore, alternative, natural non-caloric or low-caloric or reduced caloric sweeteners have been receiving increasing demand as alternatives to the artificial sweeteners and the high calorie sweeteners comprising sucrose, fructose and glucose. Like some of the artificial sweeteners, these alternatives provide a greater sweetening effect than comparable amounts of caloric sweeteners; thus, smaller amounts of these alternatives are required to achieve sweetness comparable to that of sugar. These alternative, natural sweeteners, however, can be expensive to produce and/or possess taste characteristics different than sugar (such as sucrose), including, in some instances, undesirable taste characteristics such as sweetness linger, delayed sweetness onset, negative mouth feels and different taste profiles, such as off-tastes, including bitter, metallic, cooling, astringent, licorice-like tastes.

Steviol glycosides are responsible for the sweet taste of the leaves of the stevia plant (Stevia rebaudiana Bertoni). These compounds range in sweetness from 40 to 300 times sweeter than sucrose. They are heat-stable, pH-stable, and do not ferment. They also do not induce a glycemic response when ingested, making them attractive as natural sweeteners to diabetics and others on carbohydrate-controlled diets.

The chemical structures of the diterpene glycosides of Stevia rebaudiana Bertoni are presented in FIG. 1. The physical and sensory properties are well studied generally only for Stevioside (STV) and Rebaudioside A. The sweetness potency of Stevioside is around 210 times higher than sucrose, Rebaudioside A is between 200 and 400 times, and Rebaudioside C is between 40 and 50 times and Dulcoside A around 30 times. Traditionally, Rebaudioside A is considered to have the most favorable sensory attributes of the four major steviol glycosides (see Table 1):

TABLE 1

| | | | | Optical | | | |
|---|---|---|---|---|---|---|---|
| | | | | rotation | | | |
| | | | | [a]²⁵_{D} | | | |
| | | T_{Melt} | Mol. | (H₂O, | Solubility | Relative | Quality of |
| Name | Formula | ° C. | Weight | 1%, w/v) | in water, % | sweetness | taste |
| Steviol | C₂₀H₃₀O₃ | 212-213 | 318.45 | ND | ND | ND | Very bitter |
| Steviolmonoside | C₂₆H₄₀O₈ | ND | 480.58 | ND | ND | ND | ND |
| Stevioside | C₃₈H₆₀O₁₈ | 196-198 | 804.88 | −39.3 | 0.13 | 210 | Bitter |
| Rebaudioside A | C₄₄H₇₀O₂₃ | 242-244 | 967.01 | −20.8 | 0.80 | 200-400 | Less Bitter |
| Rebaudioside B | C₃₈H₆₀O₁₈ | 193-195 | 804.88 | −45.4 | 0.10 | 150 | Bitter |
| Rebaudioside C | C₄₄H₇₀O₂₂ | 215-217 | 951.01 | −29.9 | 0.21 | 30 | Biutter |
| Rebaudioside D | C₅₀H₈₀O₂₈ | 248-249 | 1129.15 | −29.5 | 1.00 | 220 | Like sucrose |
| | | | | (ethanol) | | | |
| Rebaudioside E | C₄₄H₇₀O₂₃ | 205-207 | 967.01 | −34.2 | 1.70 | 170 | Like sucrose |
| Rebaudioside F | C₄₃H₆₈O₂₂ | ND | 936.99 | −25.5 | | ND | |
| | | | | (methanol) | | | |
| Dulcoside A | C₃₈H₆₀O₁₇ | 193-195 | 788.87 | −50.2 | 0.58 | 30 | Very bitter |
| Steviolbioside | C₃₂H₅₀O₁₃ | 188-192 | 642.73 | −34.5 | 0.03 | 90 | Unpleasant |
| Rubusoside | C₃₂H₅₀O₁₃ | ND | 642.73 | 642.73 | ND | 110 | Very bitter |

Stevia rebaudiana, after extraction and refinement is extensively used in the fields of foods, beverages, alcoholic liquor preparation, medicines, cosmetics, etc. In recent years, Stevia rebaudiana glycosides as extracts of Stevia rebaudiana have been used even more popularly as natural sweeteners and attractive alternatives to artificial sweeteners. They have become an excellent sweetening option since their caloric value is extremely low or nil and they do not cause adverse effects to dental patients and diabetic patients. The potential market is huge.

So, Stevia rebaudiana glycosides mainly comprise the following nine components: Stevioside (STV), Rebaudioside A (RA), rubusoside, dulcoside A (DA), Rebaudioside C (RC), Rebaudioside F (RF), Rebaudioside D (RD), Steviolbioside (STB), and Rebaudioside B (RB).

Steviol glycosides are characterized structurally by a single base, steviol, differing by the presence of carbohydrate residues at positions C13 and C19. They accumulate in Stevia leaves, composing approximately 10%-20% of the total dry weight. On a dry weight basis, the four major glycosides found in the leaves of Stevia typically include STV (9.1%), RA (3.8%), RC (0.6-1.0%) and dulcoside A (0.3%). RD, RB, RD, Rebaudioside E (RE), RF, Rebaudioside M (RM), STB and rubusoside.

The diterpene known as steviol is the aglycone of stevia's sweet glycosides, which are constructed by replacing steviol's carboxyl hydrogen atom with glucose to form an ester, and replacing the hydroxyl hydrogen with combinations of glucose and rhamnose to form an ether. The two primary compounds, stevioside and rebaudioside A, use only glucose: Stevioside has two linked glucose molecules at the hydroxyl site, whereas rebaudioside A has three, with the middle glucose of the triplet connected to the central steviol structure.

In terms of weight fraction, the four major steviol glycosides found in the “wild type” stevia plant tissue are:

5-10% stevioside (STV) (250-300× of sugar)

2-12% rebaudioside A (RA)—most sweet (350-450× of sugar)

1-2% rebaudioside C (RC) (40-50× of sugar)

½-1% dulcoside A. (DA)

RB, RD, RF and steviolbioside (STB) are known to be present in minute quantities;

As noted above, stevia diterpene glycosides, have a single base—steviol—and differ by the presence of carbohydrate residues at positions C13 and C19. These glycosides accumulate in Stevia leaves and compose approximately 10%-20% of the total dry weight. Typically, on a dry weight basis, the four major glycosides found in the leaves of Stevia are Dulcoside A (0.3%), Rebaudioside C (0.6%), Rebaudioside A (3.8%) and Stevioside (9.1%). Other glycosides identified in Stevia extract include Rebaudioside B, C, D, E, F, and M, Steviolbioside and Rubusoside. Among steviol glycosides only Stevioside and Rebaudioside A are currently widely available in commercial scale.

The tastes of these components are different from one another and meet different demands of different consumer populations; for example, the consumers in the United States of America and Canada are fond of RA, whereas the consumers in Japan and Korea are fond of STV.

Currently, the marketed Stevia rebaudiana glycoside products are mainly RA and STV and the methods for extracting Stevia rebaudiana glycosides also mainly focus on the purification and refinement of RA and STV.

It is generally accepted that STV may have an aftertaste which is undesirable. This aftertaste is present in Stevioside samples of even greater than 99% purity. On the other hand, RA possesses much less of an aftertaste and has a sweetness flavour comparable to sucrose. In addition to this complexity, various impurities are also present and some of these possess undesirable flavors. The entire matter is further clouded by the extreme difficulty of doing analyses.

While there is increasing commercial interest in steviol glycosides and their natural sweetening properties, there are a number of limiting factors in their use, including, for some, bitter taste, varying sweetening capabilities and extraction costs/difficulties. It is an object of the present invention to obviate or mitigate the above and other disadvantages.

### SUMMARY OF THE INVENTION

It is an object of the present invention to provide a method for extracting a composition comprising Rebaudioside M from Stevia rebaudiana with high purity, excellent color and pure taste.

The present invention further provides a natural extract comprising Rebaudioside M which is extracted and purified from the plant material according the methods described and claimed herein

The present invention further provides a natural sweetener composition comprising extracts of Rebaudioside M which are extracted and purified from the plant material as described herein. The present invention further provides a natural sweetener composition comprising a composition of Rebaudioside M extracted and purified from any of the plant material as described herein, along with at least one other steviol glycoside.

The present invention further provides foods, beverages, nutraceuticals, functional foods, medicinal formulations, cosmetics, health products, condiments and seasonings comprising compositions of Rebaudioside M extracted and purified from any of the plant material as described herein.

The natural sweetener compositions of the present invention may be zero calories or merely reduced calorie, as desired. As such, full-calorie, mid-calorie, low-calorie and zero-calorie beverages containing a Rebaudioside M extract or the sweetener compositions of the present invention are also provided.

The present invention further provides a process for extracting and purifying, from Stevia rebaudiana, a composition comprising a blend of Rebaudioside M along with at least one of Rebaudioside A, Stevioside (STV), and Rebaudioside C wherein the relative weight percent of Rebaudioside M is higher in the composition than in prior known extracted compositions.

As noted above, while Rebaudioside A has previously been considered the ideal natural sweetener, at about 300 to 450 times the sweetness of sucrose, and without any undesirable aftertaste, conventional thinking has been turned upside down more recently with the finding that Rebaudioside M shares a sweetness profile most closely aligned with sugar (see examples below), as compared to all of steviol glycosides, including RC and RA. The problem is that Rebaudioside M has a very low concentration in the plant material and leaves. The challenge is to extract and purify this glycoside.

Furthermore, the other stevia glycosides have very similar chemical structures (sharing the same diterpene skeleton and slightly differing only in the types, quantities, and structural patterns of glycoside moieties), (see FIGS. 1 and 2) so, as a result, it is difficult to obtain high purity RM at an industrial scale with commercially feasible processing costs. Due to small handling capacity and high cost, known RM extraction processes are not suitable for industrial production.

What the present invention provides are compositions of specific and selected steviol glycosides, more specifically Reb M, which achieve benefits and advantages in terms of sweetening efficacy. These natural sweetener compositions have a taste profile comparable to sugar, are desired, are not prohibitively expensive to produce and can be added, for example, to beverages and food products to satisfy consumers looking for a sweet taste. As such, these compositions allow for the customization of sweetening goals.

What the present invention provides are compositions comprising Rebaudioside M at sweetness providing/enhancing concentrations which achieve benefits and advantages above and beyond the prior extracts of steviol glycosides. These natural enhanced-Rebaudioside M based sweetener compositions have a taste profile comparable to sugar, are desired, are not prohibitively expensive to produce and can be added, for example, to beverages and food products to satisfy consumers looking for a sweet taste. As such, these compositions allow for the customization of sweetening goals. In particular, the method of extraction, provided herein, enables the cost effective and hence commercially viable production of plant extracts comprising Rebaudioside M.

The present invention provides a process for purifying Reb M including preparing a crude mother liquor/Stevia rebaudiana primary extract, passing a solution of this primary extract through a multi-column system including a plurality of columns, in series, packed with a porous adsorbent resin to provide at least one column having adsorbed Reb M and eluting fractions with Reb M content from a final column in the series of columns, said last column having absorbed Reb M to provide an eluted solution with Reb M content.

As the solution of the pre-prepared Stevia rebaudiana primary extract, comprising a plurality of steviol glycosides, passes through the multi-column system, the various glycosides separate into different portions of different columns. The portions differ from each other both by total steviol glycosides content and individual steviol glycoside content. Fractions from a final column comprising Reb M are eluted/desorbed from a final column in the multi-column system (a Reb M “eluted solution”).

Optionally, the method includes one or more additional steps. In one embodiment, the method includes washing the multi-column system with a washing solution prior to eluting fractions with Reb M content in order to remove impurities.

In another embodiment, the method optionally includes decolorizing the eluted solution with high Reb M content, removing the alcohol solvent and passing the remaining solution through a column with macroporous adsorbent to provide a second adsorption solution.

In another embodiment, the method optionally includes deionizing the eluted solution. Removal of the remaining solvent from the eluted solution—optionally decolorized and/or deionized—provides a highly purified steviol glycosides mixture comprising Reb M.

The method of the present invention also includes further processing of the eluted solution (also referred to as an intermediate Reb M extract, which is formed post passage through plurality of columns, in series, packed with a porous adsorbent resin and eluted from the final column). This intermediate Reb M extract maybe purified to remove colour, salt and impurities. This may be achieved by membrane filtration, ion exchange chromatography or activated carbon treatment (or any combinations of those).

According to one aspect of the invention, a method for producing a composition comprising a higher than conventionally extracted concentration of Reb M comprises the steps of: preparing a Stevia rebaudiana primary extract/mother liquor, suitable for passage through the porous resin columns and passing the concentrated extraction filtrate feed over a series of columns packed with macroporous resin and eluting glycosides to provide an eluate, from a final column (of said plurality of columns) containing a conventionally higher concentration of Reb M. Said eluate may be subject to one or more further purification processing steps including, but not limited to: decolorizing, evaporating, deionizing and concentrating (by, for example, nanofilters) and drying (for example via spray-drying).

These and other objects and advantages of the present invention will become more apparent to those skilled in the art upon reviewing the description of the preferred embodiments of the invention, in conjunction with the figures and examples. A person skilled in the art will realize that other embodiments of the invention are possible and that the details of the invention can be modified in a number of respects, all without departing from the inventive concept. Thus, the following drawings, descriptions and examples are to be regarded as illustrative in nature and not limiting.

### BRIEF DESCRIPTION OF THE DRAWINGS

Embodiments of the present invention will now be described, by way of example only, with reference to the attached Figures, wherein:

FIG. 1 is a molecular structure of the Reb M;

FIG. 2 are molecular structures of the diterpene glycosides of Stevia rebaudiana Bertoni wherein Glc, Xyl, and Rha represent, respectively, glucose, xylose, and rhamnose sugar moieties;

FIG. 3 is a flow diagram of the extraction process for extracting a primary extract of steviol glycosides from the leaves of Stevia rebaudiana;

FIG. 4 is a flow diagram of the purification process for purifying Reb A extract from the primary extract of steviol glycosides extracted from the leaves of Stevia rebaudiana; and

FIG. 5 is a flow diagram of the purification process for purifying STV extract from the primary extract of steviol glycosides extracted from the leaves of Stevia rebaudiana.

FIG. 6 is a bar graph showing the sweetness ratio of all the samples is tested as the concentration of 5% Sucrose solution.

FIG. 7 is a graph showing the sensory profiles for Sucrose and RM95;

FIG. 8 is a graph showing comparison of sensory profiles of RM95 and RA97

FIG. 9 shows sensory profiles for RM95, sucrose and other steviol glycosides the color depth and odor of powder;

FIG. 10 shows the time intensity of RM95, sucrose and other steviol glycosides;

FIG. 11 shows sweet releasing by time of RM95;

FIG. 12 shows sensory profiles for RM95, RA97, Sucrose and New Blends RA97&RM95;

FIG. 13 shows sensory profiles for RM95, RA97, RC85, Sucrose and New Blends RA97& RM95 & RC85;

FIG. 14 shows sensory profiles for RM95, RA97, Sucrose and New Blends RA97& RM95 & Sucrose; and

FIG. 15 shows sensory profiles for RM95, RA97, Sucrose and New Blends RA97& RM95 & Erythritol.

### DETAILED DESCRIPTION OF THE INVENTION

A detailed description of one or more embodiments of the invention is provided below along with accompanying figures that illustrate the principles of the invention. As such this detailed description illustrates the invention by way of example and not by way of limitation. The description will clearly enable one skilled in the art to make and use the invention, and describes several embodiments, adaptations, variations and alternatives and uses of the invention, including what we presently believe is the best mode for carrying out the invention. It is to be clearly understood that routine variations and adaptations can be made to the invention as described, and such variations and adaptations squarely fall within the spirit and scope of the invention.

In other words, the invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Certain definitions used in the specification are provided below. Also in the examples which follow, a number of terms are used. In order to provide a clear and consistent understanding of the specification and claims, the following definitions are provided:

The terms “an aspect”, “an embodiment”, “embodiment”, “embodiments”, “the embodiment”, “the embodiments”, “one or more embodiments”, “some embodiments”, “certain embodiments”, “one embodiment”, “another embodiment” and the like mean “one or more (but not all) embodiments of the disclosed invention(s)”, unless expressly specified otherwise.

The term “variation” of an invention means an embodiment of the invention, unless expressly specified otherwise. A reference to “another embodiment” or “another aspect” in describing an embodiment does not imply that the referenced embodiment is mutually exclusive with another embodiment (e.g., an embodiment described before the referenced embodiment), unless expressly specified otherwise.

In this specification the terms “comprise, comprises, comprised and comprising” and the terms “include, includes, included and including” are deemed to be totally interchangeable and should be afforded the widest possible interpretation.

The articles “a” and “an” are used herein to refer to one or to more than one (i.e., to at least one) of the grammatical object of the article. By way of example, “an element” means one element or more than one element.

The term “or” as used herein should be understood to mean “and/or”, unless the context clearly indicates otherwise.

The term “plurality” means “two or more”, unless expressly specified otherwise.

The term “herein” means “in the present application, including anything which may be incorporated by reference”, unless expressly specified otherwise.

The term “whereby” is used herein only to precede a clause or other set of words that express only the intended result, objective or consequence of something that is previously and explicitly recited. Thus, when the term “whereby” is used in a claim, the clause or other words that the term “whereby” modifies do not establish specific further limitations of the claim or otherwise restricts the meaning or scope of the claim.

The term “e.g.” and like terms mean “for example”, and thus does not limit the term or phrase it explains. For example, in a sentence “the image of an item is captured by an image capture device, for example a camera, the term “for example” explains that “camera” is an example of “an image capture device” through which one aspect of the data collection of this invention operates.

The term “respective” and like terms mean “taken individually”. Thus if two or more things have “respective” characteristics, then each such thing has its own characteristic, and these characteristics can be different from each other but need not be.

The term “i.e.” and like terms mean “that is”, and thus limits the term or phrase it explains.

As used herein, unless specifically indicated otherwise, the word “or” is used in the “inclusive” sense of “and/or” and not the “exclusive” sense of “either/or

The term process may be used interchangeably with method, as referring to the steps of processing as described and claimed herein.

As used herein, the term “about” in connection with a measured quantity, refers to the normal variations in that measured quantity, as expected by a skilled artisan making the measurement and exercising a level of care commensurate with the objective of measurement.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values.

Typically, steviol glycosides are obtained by extracting leaves of Stevia rebaudiana varietal, with water or alcohols (ethanol or methanol); the obtained extract is a dark particulate solution containing all the active principles plus leaf pigments, soluble polysaccharides, and other impurities. Some processes remove the “grease” from the leaves with solvents such as chloroform or hexane before extraction occurs. There are dozens of extraction patents for the isolation of steviol glycosides, such processes often being categorized by the extraction patents into those based on solvent, solvent plus a decolorizing agent, adsorption and column chromatography, ion exchange resin, and selective precipitation of individual glycosides. Methods using ultrafiltration, metallic ions, supercritical fluid extraction with CO2 and extract clarification with zeolite are found within the body of more recent patents.

At the 68th Joint Expert Committee on Food Additives (“JECFA”) meeting in 2007, steviol glycosides were defined as the products obtained from the leaves of Stevia rebaudiana Bertoni. As cited by JECFA, the typical manufacture starts with extracting leaves with hot water and the aqueous extract is passed through an adsorption resin to trap and concentrate the component steviol glycosides. The resin is washed with methanol to release the glycosides and the product is recrystallized with methanol. Ion-exchange resins may be used in the purification process. The final product is commonly spray-dried. Table 2 (at the conclusion of the disclosure) provides a product monograph of steviol glycosides, including chemical names, structures, methods of assay and sample chromatogram showing elution times of nine major glycosides.

### Method of Extraction and Purification

The present invention provides a process for extracting Reb M with high purity, concentration, excellent color and pure taste and in an amount sufficient to achieve a sweetening effect, wherein such process takes a detour from conventional extraction processes, yielding unexpected results in regards to the amount of Reb M extracted. This solution offers a clear commercial advantage given the challenges in extracting and purifying Reb M, due to its low plant concentration.

The process comprises at least two and more preferably three stages:

1) preparing a Stevia rebaudiana primary extract/mother liquor (as described herein) and measuring the concentration of Reb M therein;
2) passing the primary extract/mother liquor through a plurality of porous resin columns, in series, each of the columns packed with macroporous resin and eluting glycosides to provide an eluate from a final column (of said plurality of columns), said eluate containing a higher than conventional concentration of Reb M; and
3) purifying the eluate using further purification processing steps including, but not limited to, one or more of: decolorizing, evaporating, deionizing and concentrating (by, for example, nanofilters) and drying (for example via spray-drying).
Preparing a Stevia rebaudiana Primary Extract/Mother Liquor and Measuring the Concentration of Reb M Therein

An aspect of the present invention is the preparation and use of a particular a crude mother liquor/Stevia rebaudiana primary extract for passage through the multi-column system including a plurality of columns, in series, said column being packed with a porous adsorbent resin. It has been found, surprisingly, that a primary extract which is a by-product of a Reb A and/or STV extraction and purification process is the ideal starting material. The primary extract may be the by-product of any Reb A and/or STV extraction and purification process. In one aspect, the primary extract is the by-product of any combined Reb A and STV extraction and purification process. In another aspect, the primary extract is the by-product of process which comprises the steps of:

a) drying Stevia leaves;
b) mixing and agitating the dried Stevia leaves with water to produce a water-leaves mixture;
c) filtering the water-leaves mixture to obtain an aqueous filtrate;
d) subjecting the aqueous filtrate to ion exchange columns and adsorption resin beds to isolate a steviol glycoside extract (A);
e) isolating a Stevioside (STV) extract from the steviol glycoside extract (A), leaving steviol glycoside extract (B);
f) isolating a Rebaudioside A extract from the steviol glycoside extract (B), leaving steviol glycoside extract (C); and
g) reserving steviol glycoside extract C, said steviol glycoside extract C being a crude mother liquor/primary extract (i.e. starting material) for the purposes of the present invention.

Preferably, the mixture and agitation of the dried Stevia leaves with water is conducted with about 1 volume of water to about 10 volumes of water. Preferably, the mixture and agitation of the dried Stevia leaves with water is conducted for about one hour to about five hours at about 5° C. to about 50° C. Preferably, the water-leaves mixture is filtered to obtain an aqueous filtrate at room temperature using a compress filter. Preferably, the steviol glycoside extract is crystallized with an ethanol and/or methanol solvent to isolate and purify a Stevioside extract. Preferably, the steviol glycoside extract B is crystallized with ethanol to isolate and purify Rebaudioside A extract.

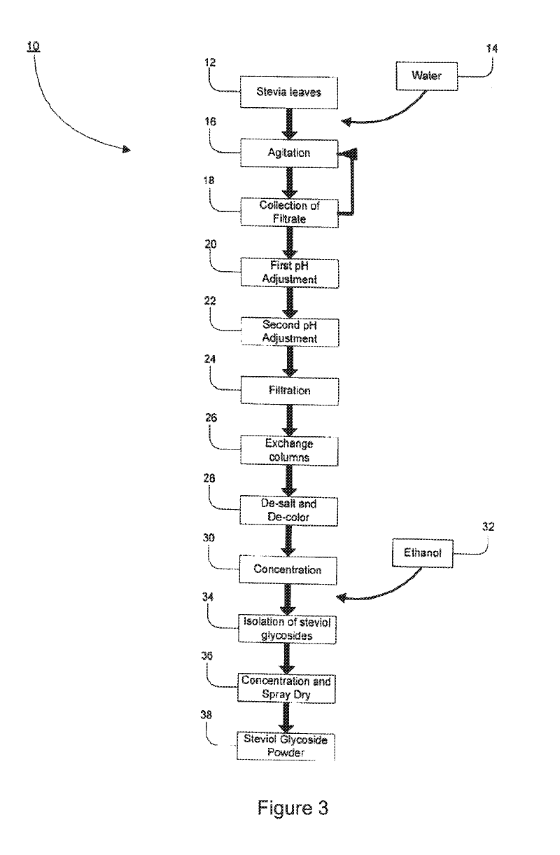

By way of further illustration and example, and not by way of limitation, FIG. 3 show the steps by which Reb A and STV extracts are isolated and the crude mother liquor/primary extract (i.e. starting material) for the purposes of the present invention may be formed. The Stevia leaves (12) are dried and the dried stevia leaves are agitated (16) in a volume of water (14) to release the sweet glycosides from the dried stevia leaves. Preferably, the sweet glycosides are released from the dried leaves using between about 1 volume to about 15 volumes of water. Even more preferably, the sweet glycosides are released from the dried leaves using about 12 volumes of water. The water-leaves mixture is agitated (16) for a period of time between about 10 minutes and about 1 hour, more preferably for a period of time between about 25 minutes and about 35 minutes. Following the agitation (16), the water-leaves mixture is drained and the filtrate collected (18). The cycle of agitation (16) and the collection of filtrate (18) is repeated for a total of about five cycles. Over the course of the five cycles, the water-leaves mixture is agitated for a total period of time between about 1 hour and about 5 hours, more preferably for a total period of time between about 2 hours and about 3 hours.

In one embodiment, for each agitation/collection cycle, the water-leaves mixture is agitated (16) in an environment having a temperature between about 5° C. and about 50° C., more preferably at a temperature between about 20° C. and about 30° C. Following the completion of the agitation/collection cycles, the pH of the water-leaves mixture is first adjusted to about pH 8.0 (20). The pH adjusted water/leaves mixture is then allowed to stand for a period of time between about 30 minutes and about two hours. The pH of the water-leaves mixture is then adjusted a second time (22) to about pH 7.0. The water-leaves mixture is subsequently filtered (24) to obtain an aqueous filtrate. The aqueous filtrate is then applied to ion exchange columns (26) to purify and decontaminate the aqueous filtrate. A person skilled in the art would understand that other methods may also be used to purify and decontaminate the aqueous filtrate. The aqueous filtrate is subsequently de-salted and de-colorized (28) and concentrated (30) using adsorption resin beds. A person skilled in the art would understand that other methods may also be used to concentrate the aqueous filtrate. A filtrate solution containing concentrated steviol glycosides is released from the adsorption resin beds (34) by rinsing the adsorption resin beds with ethanol (32), preferably about 70% ethanol (32). The filtrate solution is further concentrated and spray-dried (36) to produce a steviol glycosides containing powder (38), where the steviol glycosides include Reb A and STV. The concentration of steviol glycosides in the powder (38) varies depending on the stevia leaves (12) used, for example the concentration of RebA may be between about 24.3% to about 57.6% and the concentration of STV may be between about 24.7% to about 59.6%.

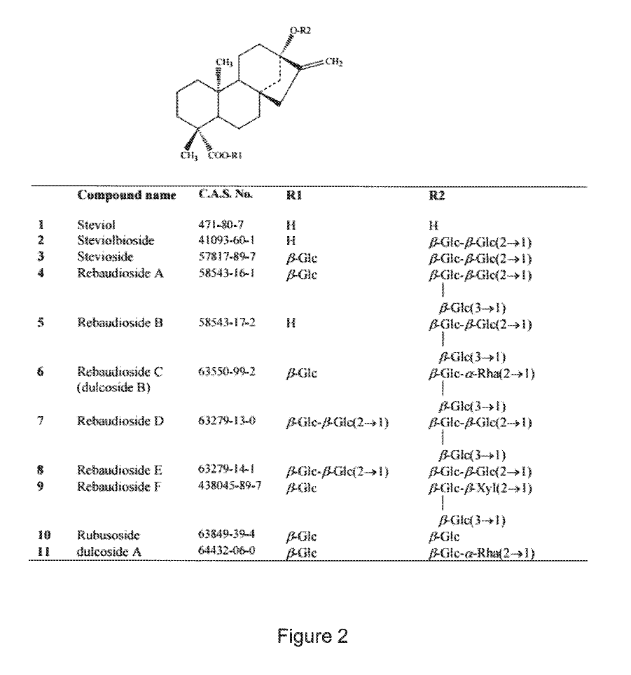

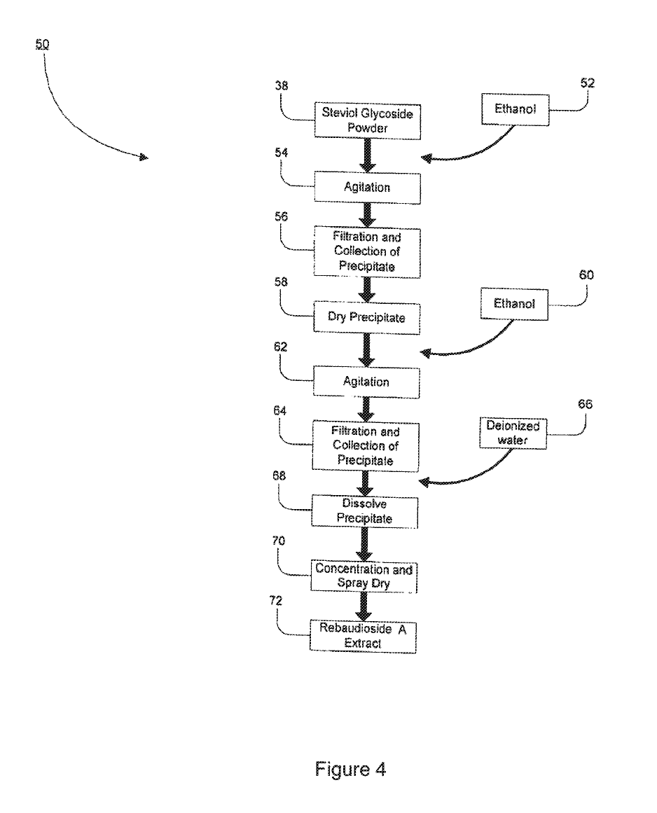

In one embodiment, Stevia leaves known to have a high content of Reb A are used to obtain a Reb A extract between about 60% and about 97.5% purity. Leaves known to have a high content of STV are used to obtain a STV extract between about 60% and about 97.5% purity. FIG. 2 illustrates a purification process (50) used to isolate Reb A extract from steviol glycoside powder (38) of FIG. 3. As shown in FIG. 4, Reb A extract is isolated using the following steps. Steviol glycoside powder (38), from the extraction process of FIG. 3, is mixed with ethanol (52), preferably between about 90% to about 95% ethanol, and the powder-ethanol mixture is agitated (54). The steviol glycoside powder (38) is mixed with preferably about two times volume (w/v) to about three times volume (w/v) of ethanol (52). Even more preferably, the steviol glycoside powder (38) is mixed with about two and a half times volume (w/v) of ethanol (52). The powder-ethanol mixture is agitated (54) for a period of time between about 30 minutes and about 2 hours, more preferably for a period of about one hour.

In one embodiment, the powder-ethanol mixture is agitated (54) in an environment having a temperature between about 25° C. and about 60° C., more preferably at a temperature between about 45° C. and about 50° C. The powder-ethanol mixture is subsequently filtered and the precipitate is collected (56). The precipitate is then dried (58). The precipitate is then mixed with ethanol (60). The ethanol (60) mixed with the precipitate is preferably between about 90% to about 95% ethanol, more preferably about 92% ethanol. Preferably, the precipitate is mixed with between about two times volume (w/v) to about four times volume (w/v) of ethanol (60). Even more preferably, the precipitate is mixed with three times volume (w/v) of ethanol 60. The precipitate-ethanol mixture is slowly agitated (62) for a period of time between about 45 minutes and about 1 hour, more preferably for a period of about 50 minutes.

In one embodiment, the precipitate-ethanol mixture is agitated (62) in an environment having a temperature between about 25° C. and about 60° C., more preferably at a temperature between about 45° C. and about 50° C. Following agitation (62) of the precipitate-ethanol mixture, the precipitate-ethanol mixture is filtered and the precipitate is collected (64). The precipitate comprises crystals of RebA, preferably crystals of higher purity Reb A, even more preferably crystals of about 95% Reb A content. The precipitate is subsequently dissolved (68) in deionized water (66). The solution is then concentrated and spray-dried (70) to produce a final Reb A extract (72).

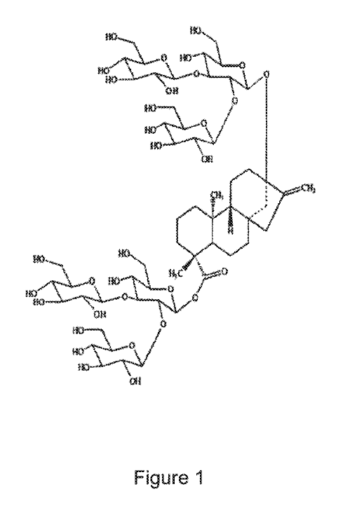

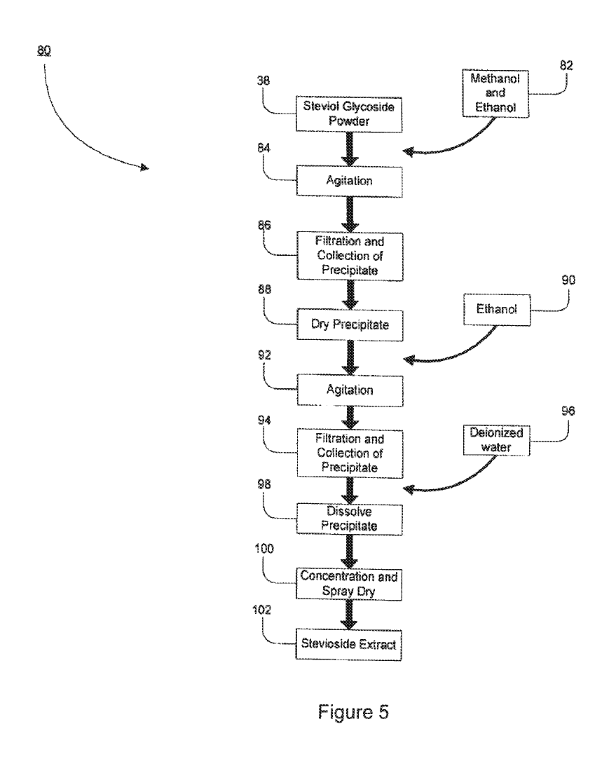

In one embodiment, the Reb A extract (72) is about 97.5% purity. A person skilled in the art would understand that other methods may also be used to dry the precipitate. FIG. 5 illustrates a purification process (80) used to isolate STV extract from the steviol glycoside powder (38) of FIG. 1. As shown in FIG. 3, STV extract is isolated using the following steps. Steviol glycoside powder (38) is mixed with a mixture of methanol and ethanol (82). The ratio of methanol to ethanol in the methanol-ethanol mixture (82) is preferably about 4:1. Preferably, the steviol glycoside powder (38) is mixed with between about two times volume (w/v) to about four times volume (w/v) of the methanol-ethanol mixture (82). Even more preferably, the steviol glycoside powder (38) is mixed with about three times volume (w/v) of the methanol-ethanol mixture (82). The powder-methanol-ethanol mixture is agitated (84) for a period of time between about 30 minutes and about 2 hours, more preferably for a period of about one hour.

In one embodiment, the powder-methanol-ethanol mixture is agitated (84) in an environment having a temperature between about 25° C. and about 60° C., more preferably at a temperature between about 45° C. and about 50° C. The powder-methanol-ethanol mixture is subsequently filtered and the precipitate is collected (86). The precipitate is the dried (88). The precipitate is then mixed with ethanol (90). The ethanol (90) that is mixed with the precipitate is preferably between about 87% to about 95% ethanol, more preferably about 90% ethanol. Preferably, the precipitate-ethanol mixture is mixed with about one and a half times volume (w/v) to about two and half times volume (w/v) of ethanol (90). Even more preferably, the precipitate-ethanol mixture is mixed with two times volume (w/v) of ethanol (90). The precipitate-ethanol mixture is slowly agitated (92) for a period of time between about 45 minutes and about 1 hour, more preferably for a period of about 50 minutes.

In one embodiment, the precipitate-ethanol mixture is agitated (92) in an environment having a temperature between about 25° C. and about 60° C., more preferably at a temperature between about 45° C. and about 50° C. Following agitation (92) of the precipitate-ethanol mixture, the precipitate-ethanol mixture is filtered and the precipitate is collected (94). The precipitate comprises crystals of STV, preferably crystals of higher purity STV, even more preferably crystals of about 95% STV content. The precipitate is subsequently dissolved (98) in deionized water (96). The solution is then concentrated and spray-dried (100) to produce a final STV extract (102).

In one embodiment, the STV extract (102) is about 97.5% purity. A person skilled in the art would understand that other methods may also be used to dry the precipitate. Following the extraction process (10) shown in FIG. 1 and purification of Reb A extract (72) and STV extract (102), the Reb A extract (72) and STV extract (102) are blended for use in natural sweetener compositions. The sweetener compositions described above are: (a) low calorie or reduced calorie; (b) made from all natural products; (c) have a favourable safety profile; (d) demonstrate good thermal stability during processing; and (e) are less fermentable by oral dental-caries causative microorganisms than sugar.

In one embodiment, the Stevia rebaudiana plant material (e.g. leaves) may be dried at temperatures between about 20° C. to about 60° C. until a moisture content between about 8% and about 12% is reached. In a particular embodiment, the plant material may be dried between about 20° C. and about 60° C. for a period of time from about 1 to about 24 hours, such as, for example, between about 6 to about 12 hours. In other particular embodiments, the plant material may be dried at temperatures between about 40° C. to about 45° C. to prevent decomposition.

In some embodiments, the dried plant material is optionally milled. Particle sizes may be between about 10 to about 20 mm.

Before further processing of the Stevia rebaudiana primary extract/mother liquor, the concentration therein of Reb M is measured, for example, by liquid chromatographic analysis (for example, HPLC). It is desired for the Reb M concentration in the mother liquor be greater than 1% by dry weight. It is further desired that the Reb M concentration in the mother liquor be greater than 2% by dry weight. The higher Reb M content in the mother liquor, the better, however commercially and practically, within the scope of the invention, the target is an enriched Reb M content of about 2.3% by dry weight.

For greater clarity in regards to the starting material, the “mother liquor” is the by-product of a crystallization of an RA, an STV or a combined RA/STV process and as such is generally spray dried, forming a dried powder. This dried powder “mother liquor” is then dissolved into a solvent (preferably water) and preferably with agitation to prepare the solution suitable for feeding into the plurality of resin columns in series, such solution comprising a mother liquor (solids) content of about 15-30 m/L, more preferably 20-30 m/L, even more preferably 23-26 g/L and most preferably 25 g/L. Also, to be clear, the measured Reb M concentrations, referred to above, are the Reb M concentrations in the mother liquor (i.e. dried powder) tested by liquid chromatography analysis.

Preferably, the steviol glycoside mother liquor used in the process of this invention refers to the byproduct of steviol glycosides from which RA and STV have been extracted and having a measured RM content of 2% or higher. It may be produced from stevia extract or other stevioside products.

Passing the Primary Extract/Mother Liquor Through a Plurality of Porous Resin Columns, in Series, Each of the Columns Packed with Macroporous Resin and Eluting Glycosides to Provide an Eluate from a Final Column (of Said Plurality of Columns), Said Some of the Eluate Fractions Containing a Higher than Conventional Concentration of Reb M

The method of the invention comprises a method for extracting and purifying Reb M including passing a solution of a pre-prepared Stevia rebaudiana primary extract (the final prepared solution, as noted above), through a multi-column system including a plurality of columns, in series, packed with a porous adsorbent resin to provide a final column having adsorbed Reb M and eluting fraction with high Reb M content from the last column to provide an eluted solution with Reb M content (the “eluted Reb M extract”), such eluted Reb M extract available for optional downstream purification steps.

In another aspect of the invention, a method for purifying Reb M comprises:

(a) passing the pre-prepared Stevia rebaudiana primary extract (the final prepared solution, as noted above) through a multi-column system including a plurality of columns in series packed with a porous adsorbent resin to provide at least one column having adsorbed glycosides; and
(b) eluting fractions with Reb M content from the final column having adsorbed steviol glycosides to provide an eluted solution fractions comprising Reb M.

In another aspect of the invention, a method for extracting and purifying Reb M comprises:

(a) passing the pre-prepared Stevia rebaudiana primary extract (the final prepared solution, as noted above), through a multi-column system including a plurality of columns in series packed with an adsorbent resin to adsorb steviol glycosides;
(b) eluting Reb M from the last (final) column; and
(c) removing impurities from the multi-column system.

In another embodiment, a method for extracting and purifying Reb M comprises:

(a) passing the he pre-prepared Stevia rebaudiana primary extract (the final prepared solution, as noted above), through a multi-column system including a plurality of columns in series packed with an adsorbent resin to provide at least one column having adsorbed glycosides;
(b) eluting fractions with Reb M content from the last column having adsorbed glycosides to provide an eluted solution with Reb M content;
(c) decolorizing the eluted solution to provide a first adsorption solution; and
(d) removing the alcoholic solvent from the first adsorption solution and passing the remaining solution through a column with a macroporous adsorbent to provide a second adsorption solution.

In another embodiment, a method for extracting and purifying Reb M comprises:

(a) passing the he pre-prepared Stevia rebaudiana primary extract (the final prepared solution, as noted above), through a multi-column system including a plurality of columns in series packed with an adsorbent resin to provide at least one column having adsorbed glycosides;
(b) removing impurities from the multi-column system;
(c) eluting fractions with Reb M content from the final column having adsorbed glycosides to provide an eluted solution with Reb M content;
(d) decolorizing the eluted solution with Reb M content to provide a first adsorption solution; and
(e) removing the alcoholic solvent from the first adsorption solution and passing the remaining solution through a column with a macroporous adsorbent to provide a second adsorption solution.

In another embodiment, a method for purifying Reb M comprises:

(a) passing a he pre-prepared Stevia rebaudiana primary extract (the final prepared solution, as noted above), through a multi-column system including a plurality of columns in series packed with an adsorbent resin, to provide at least one column having adsorbed glycosides;
(b) eluting fractions with Reb M content from the last column having adsorbed glycosides to provide an eluted solution with Reb M content; and
(c) deionizing the eluted solution.

As the solution of the pre-prepared Stevia rebaudiana primary extract, comprising a plurality of glycosides but with a specific pre-selected concentration of Reb M, passes through the multi-column system, the various steviol glycosides separate into different portions of different columns. The portions differ from each other both by total steviol glycosides content and individual steviol glycoside content. Fractions containing high Reb M content are eluted/desorbed from the multi-column system separately from fractions containing low Reb M content concentrated extraction filtrate

More specifically, the method comprises separating the intermediate Reb M extract/composition by passage of the concentrated extraction filtrate through a plurality of resin adsorption columns, in series connection, to form a separated filtrate that is the desired intermediate Reb M extract.

A key to the numerous advantages of the process described herein is the passage of the Stevia rebaudiana primary extract through a multi-column system including a plurality of columns, in series, packed with a porous adsorbent resin and the elution of fractions with Reb M content and removal of an eluate from the final column only, said eluate comprising the desired intermediate Reb M extract, available for further optional downstream purification steps.

In this way, a solution of the primary extract may be passed through one or more consecutively connected columns, connected serially, and packed with macroporous polymeric adsorbent (preferably polar) to provide at least one column having adsorbed glycosides. In some embodiments, the number of columns can be six, in others, the number of columns may be seven, in others, the number of columns may be eight. Most preferably, there are eight columns in series—which is optimal if the concentration of Reb M in the mother liquor (i.e. dried powder) tested by liquid chromatography analysis is 2-3 g/L. Most preferably, there are 6-7 columns in series—which is optimal if the concentration of Reb M in the mother liquor (i.e. dried powder) tested by liquid chromatography analysis is around over 3.0 g/L. Most preferably, there are ten columns in series—which is optimal if the concentration of Reb M in the mother liquor (i.e. dried powder) tested by liquid chromatography analysis is less than 2.0 g/L. In other words, it is clear and has been found that the concentration of Reb M in the mother liquor (i.e. dried powder) tested by liquid chromatography analysis directly dictates the number of columns to employ.

In certain embodiments, the first column in the sequence can be a “catcher column”, which is used to adsorb certain impurities, that have higher adsorption rates and faster desorption rates than most glycosides. In some embodiments, the “catcher column” size can be about one-third the size of the remaining columns. The ratio of internal diameter to column height or so-called “diameter: height ratio” of the columns may be between about 1:1 to about 1:100, such as, for example, about 1:2, about 1:6, about 1:10, about 1:13, about 1:16, or about 1:20. In a particular embodiment, the diameter: height ratio of the column is about 1:3. In yet another embodiment, the diameter: height ratio is about 1:8. In still another embodiment, the diameter: height ratio is about 1:10

The polar macroporous polymeric adsorbent may be any macroporous polymeric adsorption resins capable of adsorbing glycosides, such as, for example, the Amberlite® XAD series (Rohm and Haas), Diaion® HP series (Mitsubishi Chemical Corp), Sepabeads® SP series (Mitsubishi Chemical Corp), Cangzhou Yuanwei YWD series (Cangzhou Yuanwei Chemical Co. Ltd., China), DA-201-H or the equivalent. In a most preferred form, the column is a macroporous adsorption resin type: D201-H (Jiangsu Suqing Ltd), with a specific surface area of 800 m²/g and average pore size: 6 to 8 nm. The individual columns may be packed with the same resin or with different resins. The columns may be packed with sorbent up to from about 75% to about 100% of their total volume.

The solvent that carries the steviol glycoside solution through the column system may comprise water, alcohol or a combination thereof (for example, an aqueous alcoholic solvent). The water to alcohol ratio (vol/vol) in the aqueous alcoholic solvent may be in the range of about 99.9:0.1 to about 60:40, such as, for example, about 99:1 to about 90:10. The specific velocity (SV) can be from about 0.3-1 to about 1.5-1, such as, for example, about 1.0 hour-1. Preferably, alcohol is selected from the group consisting of methanol, ethanol, n-propanol, 2-propanol, 1-butanol, 2-butanol and mixtures thereof.

The alcohol can be selected from, for example, methanol, ethanol, n-propanol, 2-propanol, 1-butanol, 2-butanol and mixtures thereof.

Glycosides contained with the solution of the primary extract become adsorbed with the pores of the selected resin, packed inside the columns upon passage of the solution through the plurality of columns, in series. Desorption, i.e. release of the trapped glycosides, can be carried out with an aqueous alcohol solution. Suitable alcohols include methanol, ethanol, n-propanol, 2-propanol, 1-butanol, 2-butanol and mixtures thereof. In a particular embodiment, the aqueous alcoholic solution can contain between about 30% to about 70% alcohol content, such as, for example, between about 40% to about 70%, about 50% to about 65%, about 58%, about 59%, about 60%, about 65%, about 70%. In a particular embodiment, the aqueous alcoholic solution contains between about 55% to about 75% ethanol. A SV between about 0.5 hour-1 to about 3.0 hour-1, such as, for example, between about 1.0 hour-1 and about 1.5 hour-1 can be used.

During desorption/elution from the end of the series connected columns, samples are periodically taken (for example 100 ml to 500 ml at a time) and are tested/analyzed for sweetness to determine when the columns are “clean” of the desired glycosides, and in particular Reb M.

The resin columns can be regenerated and reused. Upon complete passage through the one or more columns, the resins can optionally be washed with a washing solution to remove impurities. Suitable washing solutions include an aqueous or alcoholic solution, where the aqueous solution can contain any suitable acid or base to arrive at the desired pH.

The water to alcohol ratio (vol/vol) in the aqueous alcoholic solution is in the range of about 99.9:0.1 to about 60:40. Multiple washes of the columns with the same, or different, wash solutions can be performed, followed by wash(es) with water until the pH of the effluent from the one or more columns is about neutral (i.e., has a pH from about 6.0 to about 7.0). In a particular embodiment, the resins of the one or more columns is washed sequentially with one volume of water, two volumes of NaOH, one volume of water, two volumes of HCl, and finally with two volumes of water until it reached a neutral pH. The elution of impurities is carried out from two or more consecutively connected columns, as they are provided serially.

The Reb M i) concentration in the mother liquor and also ii) elution from the final column may be determined experimentally by HPLC or HPLC/MS. For example, chromatographic analysis can be performed on a HPLC/MS system comprising an Agilent 1200 series (USA) liquid chromatograph equipped with binary pump, autosampler, thermostatted column compartment, UV detector (210 nm), and Agilent 6110 quadrupole MS detector interfaced with Chemstation data acquisition software. The column can be a “Phenomenex Prodigy 5u ODS3 250×4.6 mm; 5 μm (P/No. 00G-4097-E0)” column maintained at 40° C. The mobile phase can be 30:70 (vol/vol.) acetonitrile and water (containing 0.1% formic acid) and the flow rate through the column can be 0.5 mL/min.

A plurality of macroporous resin chromatographic columns, in series connection, are used for separation. More preferably, a macroporous adsorption resin is mounted into the resin column, with resin columns in series connection constituting a “resin column set”, and the feed liquor passes through the columns for adsorption. After adsorption, resolution is made with alcohol (for example 60-75% of ethanol) and the resolving solution is collected. In this resolving process, resolutions are conducted by stages based on the volume of solution collected and an analysis is made to respectively for each fraction. The next processing procedure corresponding to the resolving solution will be based on the content of Reb M in the solution. As such, the eluted fraction of the final column is analyzed for the solid content, the steviol glycosides content and Reb M content. If the Reb M content is above a desired amount (such as, for example, above-20%), the next step process will be continued. It has found that eight columns connected in series is the most efficient way to achieve a desired 20-35 wt % of Reb M.

At this step and critically, multiple sets of said resin columns are connected in series, each set acting to absorb in order from the first column to the fourth column until the eluant of the last (preferably the eighth) column releases an extract/solution with the desired sweetness. Adsorption between the sets is subject to selective adsorption and chromatographic separation based on product requirements.

It is most preferred that the feed liquor to upper column has a flow range of 2-4 L/min until the effluent water becomes colorless.

What preferable is, after water washing, 70% alcohol is used for resolution, and the resolving solution with alcohol content ≧50% is collected; the described resolution by stages means: when the resolving solution carrying particles of different size passes through the resin, the particles of different size pass through the resin along with the leakage solution at different periods, the leaking solution at different periods is resolved respectively, and HPLC analysis is performed on the resolving solution collected at all stages.

As is clear herein, the method for purification of Reb M, as described and claimed herein is divided into i) preparation of mother liquor/primary extract; ii) preparation of crude extract by passage through multiple resin columns, in series, with particular preferable conditions; and iii) subsequent purification. The crude extraction comprises: feed liquor flows through preferably eight macroporous resin columns in series to be adsorbed in order; wherein alcohol is resolved by stages and analyzed; the eluent is concentrated under reduced pressure at a high temperature; and wherein the resulting solid is dried in vacuum. A crude Reb M composition with mass content up to 25-35% is produced. The subsequent purification steps comprise: heating a mixed solvent; dissolving the crude Reb M in a mixed solvent to form a mixture and then cooling the mixture to room temperature; (preferably stirring at intervals); separating solids-liquids after a standing period (preferably 2-3 hours), drying the resulting solid to produce purified Reb M with a mass content up to over 95%. It has been found that Reb M has more than 300 times sweetness than sucrose and is more durable than sucrose, with no lingering bitterness and closer taste to sucrose in good taste quality, so it is an ideal natural additive sweetener.

In one aspect, the crude extraction comprises the following steps: the steviol glycoside mother liquor—dissolving the spray dried mother liquor in to water) is prepared into 20-35 g/L feed liquor; the described feed liquor flows through the macroporous resin column at a rate of 2.0-4.0 L/min, eight resin columns are connected in series, the feed liquor upper column is absorbed through the eight columns in order until the last column effluent becomes sweet, washed with water, after the end of adsorption, resolution is made with alcohol of 70%-77% mass concentration, resolving solution is collected, and in this resolving process, resolution is conducted by stages based on the volume of solution leakage, and the 1,100-1,300 L eluent is tested with high performance liquid chromatography (HPLC); the 1,100-1,300 L eluent is concentrated under reduced pressure at a temperature of 60-80° C., and the resulting solid is dried in vacuum; and a crude Steviol Glycoside Reb M with mass content up 25-35% is produced.

Wherein the said macroporous resin column is styrene-type polar copolymer, and the described macroporous resin column has an average pore size of 6-8 nm A, pore volume 0.9-1.0 ml/g and PH value of 4.5-5.5 during adsorption;

Preferably, the described macroporous resin column has the particle size range of 16-60 mesh. Preferably, the described macroporous resin column has the specific surface area of 1,300-1,400 m2/g. Preferably, the described macroporous resin column has the moisture content of 65%-75%. Preferably, the described macroporous resin column has the wet bulk density of 0.65-0.70 g/ml.

After the end of described adsorption, it is advisable to use the alcohol with mass concentration of 75%-77%.

Preferably, the type of DA201-H macroporous resin is selected and used for adsorption, the surface properties of the macroporous resin is low pole. In the elution step after adsorption, the mass concentration of alcohol directly affects the content of Reb M in eluent. In the Reb M purification method of this invention, the preferred concentration of alcohol is 75%-77%. In the subsequent purification process, the cooling time during cooling have an impact on the crystallization effect of crystalline liquid, it is preferable to cool the mixed liquor down to room temperature within 25 minutes.

Purifying the Eluate Using Further Purification Processing Steps Including, but not Limited to, One or More of: Decolorizing, Evaporating, Deionizing and Concentrating (by, for Example, Nano-Filters, or Evaporators) and Drying (for Example Via Spray-Drying)

The method of the present invention also includes further optional processing of the intermediate Reb M extract (the product of the final column). The intermediate Reb M extract may, at the least, be purified to remove colour, salt and impurities. This may be achieved, for example, by membrane filtration, ion exchange chromatography or activated carbon treatment (or any combinations of those).

Preferably, the intermediate Reb M extract (a separated filtrate) is purified and then concentrated to form a concentrated separated filtrate. By way of example, the concentrated separated filtrate maybe further purified in a gel column to be remove impurities and homologs of Reb M. The resulting product from the gel column may be concentrated and/or spray dried to a final purified product.

Wherein compounds are required to be separated in accordance with the method of the invention, separation can be achieved by any suitable means including, but not limited to, gravity filtration, a plate-and-frame filter press, cross flow filters, screen filters, Nutsche filters, belt filters, ceramic filters, membrane filters, microfilters, nanofilters, ultrafilters or centrifugation. Optionally various filtration aids such as diatomaceous earth, bentonite, zeolite etc., may be used in this process.

Wherein solutions are to be treated by ion exchange resins in accordance with the method of the invention, such deionization by any suitable method including, for example, filtration (nano- or ultra-filtration), reverse osmosis, ion exchange, mixed bed ion exchange or a combination of such methods.

The cation-exchange resin can be any strong acid cation-exchanger where the functional group is, for example, sulfonic acid. Suitable strong acid cation-exchange resins are known in the art and include, but are not limited to, Rohm & Haas Amberlite® 10 FPC22H resin, which is a sulfonated divinyl benzene styrene copolymer, Dowex® ion exchange resins available from Dow Chemical Company, 15 Serdolit® ion exchange resins available from Serva Electrophoresis GmbH, T42 strong acidic cation exchange resin and A23 strong base an ion exchange resin available from Qualichem, Inc., and Lewatit strong ion exchange resins available from Lanxess. In a particular embodiment, the strong acid cation-exchange resin is Amberlite® 10 FPC22H resin (H+). As would be known to those skilled in the art, other suitable strong acid cation-exchange resins for use with embodiments of this invention are commercially available.

The anion-exchange resin can be any weak base anion-exchanger where the functional group is, for example, a tertiary amine. Suitable weak base anion exchange resins are known in the art and include, but are not limited to, resins such as Amberlite-FPA53 (OH—), Amberlite IRA-67, Amberlite IRA-95, Dowex 67, Dowex 77 and Diaion WA 30 may be used. In a particular embodiment, the strong acid cation-exchange resin is Amberlite-FPA53 (OH—) resin. As would be known to those skilled in the art, other suitable weak base anion-exchange resins for use with embodiments of this invention are commercially available.

Those of skill in the art will also recognize that one or more of the “decolorizing”, “second adsorption” and “deionization” steps, described herein may be omitted. Those experienced in art will also understand that although the process described herein assumes certain order of the described steps, this order can be altered in some cases.

At this stage, colour salt and other impurities may optionally be removed by a variety of methods including via ion exchange resins, membrane filtration and activated carbon treatment

A selected acidic cation exchange resin—001×16 and basic anion exchange resin—D301R may be mounted into the resin column respectively. The resolving solution requiring anion and cation exchange as determined in the step above are treated by the anion and cation exchange resin respectively. The purified solution from the anion and cation exchange resin columns is collected. After water washing, all effluent solutions are merged. Preferably, the acidic cation exchange resin is 001×16 benzene ethylene strongly acidic cation, basic anion exchange resin is D301R strongly basic anion, and the flow of resolving solution in processing is =2˜3BV/h.

Using the special anion and cation resins is preferred for purification. While other resins may also work, anion and cation resins are most efficient for this process. This step remove the colors, slats and improve the sensory profile of the final product.

### Concentration and Drying:

The resulting material may be dried by using a conventional spray drying unit or by using a conventional spray agglomeration unit, rotary evaporation, spray drying or other means. Or the material previously prepared may be used as-is.

In one aspect, the solution may proceed through dynamic dealcoholization and dewatering and concentration by organic membranes, the secondary concentration, reduced-pressure distillation and concentration to a nominal concentration of 40-50% solid content and finally spray drying to come to the final product, that is, the composition comprising Reb M (the Reb M extract).

The resolving solution resulting from above steps is then concentrated at reduced vacuum at a temperature <70° C. into concentrated solution from which alcohol (for example ethanol) is recovered, wherein the concentrated solution is concentrated to be, preferably, ______.

### Purification

Preferably, after the spray drying, the spray dried powder is dissolved into an alcohol solution (for example ethanol or methanol and most preferably a blend of ethanol/methanol) and water solvent for crystallization. This may be achieved in a variety of ways and known and appreciated in the art.

In one aspect, the subsequent purification process may comprise the following steps: alcohol with volume concentration of 90±2% and 88±2% isopropyl alcohol are fully mixed into a mixed solvent by ratio 3:2, and heated to 65-75° C.; the described crude Steviol Glycoside Reb M is put in the mixed solvent with a mass ratio to the crude Steviol Glycoside Reb M of 3.0-3.5:1; Steviol Glycoside Reb M is dissolved in the mixed solvent to form a mixture, then cooled to normal temperature in 25 minutes; and then allowed to stand, and stirred at regular intervals during standing; after standing 48-60 hours, solid-liquid separation is conducted and the resulting solid is dried to produce purified stevioside RM with content more than 95%.

In the subsequent purification process, drying of the solid from solid-liquid separation means that solid is dissolved in desalted water to form a solution with mass concentration of 25±2%, then the solution is concentrated to 45±2%, after which the solution is concentrated and dried to purified Steviol Glycoside Reb M content of more than 95%;

The method is characterized in that: in the subsequent purification process, the mixed solvent is preferably heated to 67-72° C.

Compared with the existing technologies, the method of the invention has the following advantages and effects: (1) selective adsorption: the macroporous resin column acts to absorb in the form of (preferably eight) resin columns connected in series into a set, and multiple sets of resin column connected which allows the actual operation to be free from influence of external factors such as periodic cleaning, regeneration and raw and auxiliary materials, in this way, production efficiency can be improved, service life of resin can be extended, and product quality can be ensured stably. (2) Collection of resolving solution by stages: eluting solution is collected by stages, and merged for processing in batches depending on the content of Reb M, rather than the usual way of all resolving solution being collected together for processing. In this way, separation of the high-Reb M batches and medium and low-Reb M batches of products can be ensured so as to guarantee product quality and shorten the process cycle, as well as provide corresponding products selectively according to the needs. (3) Significant integrative effect: the purification process of this invention achieves the effect of enrichment by stages for a variety of glycosides in bed, and the extracted Reb M has high purity, excellent color and pure taste.

Within the scope of the present invention, it is preferred to extract and purify a composition comprising Reb M. The Reb M content in the final product will most preferably reach up to 95 wt % by dried weight. This content is considered an upper ideal. Achieving 95 wt % or 97 wt % is possible using the method of the invention but the cost-benefit analysis suggests that this content is commercially viable and achieves the desired sweetening. If cost is no issue in its application, the method of the invention could achieve over 99 wt % content of Reb M. The Reb M content is the most important factor for the product sensory taste profile. The focus of the method of the invention is to economically and practically extract Reb M. It has been found that when the Reb M content extracted is at about 95 to 97 wt %, the total glycosides content in the extracted product is about 97-98 wt %. In this way, a highly desirable, commercial product is created. So, while the term “high” is used herein in regards to Reb M content, it is intended also to encompass an extract/composition which comprises a Reb M content of from about 90-99 wt %, in one preferred form.

In one embodiment, separation produces a composition comprising greater than about 95% by weight of the Reb M “target” steviol glycoside on an anhydrous basis, i.e., a highly purified steviol glycoside composition. In another embodiment, separation produces a composition comprising greater than about 96% by weight of the Reb M target steviol glycoside. In particular embodiments, the composition comprises greater than about 97% by weight of the Reb M target steviol glycoside. In other embodiments, the composition comprises greater than about 99% by weight of the Reb M target steviol glycoside.

The Reb M target steviol glycoside can be in any polymorphic or amorphous form, including hydrates, solvates, anhydrous or combinations thereof.

Compared with the existing technologies, this invention has the following advantageous effects: the Reb M purification process of this invention includes the crude extraction and subsequent purification of raw materials. In the purification process, crude steviol glycoside Reb M with mass content of more than 25-35% and purified steviol glycoside Reb M with content of more than 95% are produced by choosing macroporous resin column material with suitable polarity, specific surface area, average pore size and pore volume; setting preferable feed liquor concentration and pH value, as well as the concentration of alcohol, accurate control of dissolution temperature and precise preparation of the proportioning of solvent, etc. a number of technical measures. Based on the qualitative and quantitative analysis if the resulting RM sample, it is confirmed that this component has indeed advantages that other components cannot substitute, for example: it has more than 300 times sweetness of sucrose, and is more durable than sucrose, with no lingering bitterness and closer taste to sucrose in good taste quality, so it is an ideal natural additive sweetener.

Statistics show that the content of Steviol Glycoside Reb M is very low stevia leave, in generals less than 0.1%. Because of the low content, there are some technical difficulties in both analysis and testing and extraction of RM. The purification method of this invention overcomes the defects in existing technologies such as differences in the purification process, quality stability, yield and content, and produces the objective product of high purity Steviol Glycoside Reb M which is industrially disadvantageous. This invention provides Reb M-based Steviol Glycoside product to meet the different needs of consumers.

### Natural Sweetener Compositions

Natural sweetener compositions of the present invention comprise the Reb M extract, described and claimed herein. This Reb M extract has a taste profile comparable to sugar and may be blended into a variety of natural sweetener compositions. Such a composition can be added, for example, to beverages and food products to satisfy consumers looking for a sweet taste. There is provided herein a process to selectively extract Reb M from fresh stevia plants in a manner which reduces negative properties in order to customize sweetening goals.

### Formulations

A further aspect of the present invention provides a solution to the problem of reduction of sugar intake while not sacrificing sweet taste. The present invention takes full advantage of the appreciated properties of RM, while creating an extract free or substantially free of bitter taste, and which can be produced without extraction/cost difficulties. The present invention not only overcomes the disadvantages of high calories and health effects due to excessive intake of white sugar, but also utilizes fully the advantage of RM in being purely natural, and having a high sweetness, and good safety and stability; and the compounded sweetener has a better mouth-feel and fresher taste, and is safer and more convenient for use, meeting people's demands for reducing calories in diets.

Composition of the invention, comprising extracted and purified RM, prepared in accordance with the present invention, may be used in a variety of consumable products including, but not limited to, foods, beverages, pharmaceutical compositions, tobacco products, nutraceutical compositions, oral hygiene compositions, and cosmetic compositions.

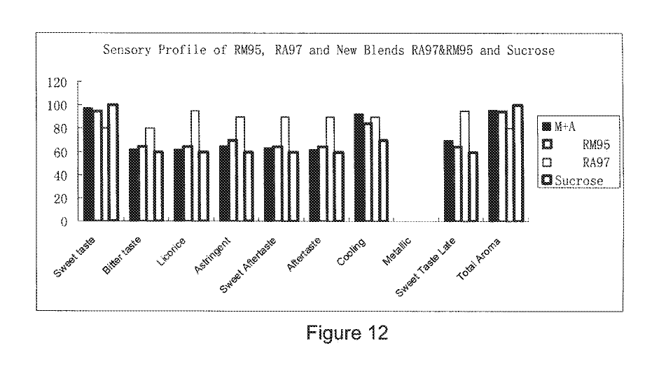

The RM obtained in this invention, having a molecular weight of 1291.29, a molecular formula of C56H90033, CAS registry number 1220616-44-3, and the structure presented in FIG. 2, is in the form of a white and odorless powder. The compound is about 200 times sweeter than sugar when compared to a 10% sucrose solution. The infrared absorption spectrum is shown in FIG. 12.

Other properties of RM include a melting point of 249-250° C., and a specific rotation of [α]D 25-19.0° in 50% ethanol (C=1.0). The solubility of RM in water is around 0.3%, and increases with an increase in temperature.

RM is soluble in diluted solutions of methanol, ethanol, n-propanol, and isopropanol. However, it is insoluble in acetone, benzene, chloroform, and ether.

RM obtained in accordance with the present invention is heat and pH-stable.

Composition of the invention, comprising extracted and purified RM, obtained according to this invention can be used “as-is” or in combination with at least one sweetener, flavor, food ingredient and/or combination thereof.

Non-limiting examples of flavors include lime, lemon, orange, fruit, banana, grape, pear, pineapple, mango, berry, bitter almond, cola, cinnamon, sugar, cotton candy and vanilla flavors and/or combination thereof.

Non-limiting examples of other food ingredients include at least one selected from flavors, acidulants, organic and amino acids, coloring agents, bulking agents, modified starches, gums, texturizers, preservatives, antioxidants, emulsifiers, stabilizers, thickeners and gelling agents and/or combination thereof.

Composition of the invention, comprising extracted and purified RM, prepared in accordance with the present invention, may be incorporated as a high intensity natural sweetener in foodstuffs, beverages, pharmaceutical compositions, cosmetics, chewing gums, table top products, cereals, dairy products, toothpastes and other oral cavity compositions, etc.

Composition of the invention, comprising extracted and purified RM, prepared in accordance with the present invention, as a sweetening compound may be employed as the sole sweetener, or it may be used together with at least one naturally occurring high intensity sweeteners such as stevioside, RA, RB, RC, RD, RE, RF, steviolbioside, dulcoside A, rubusoside, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobtain, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, Luo Han Guo sweetener, mogroside V, siamenoside and/or combination thereof.

In a particular embodiment, the composition of the invention, comprising extracted and purified RM, prepared in accordance with the present invention, can be used together in a sweetener composition comprising a compound selected from the group consisting of RA, RB, RD, NSF-02, Mogroside V, erythritol and/or combinations thereof.

Composition of the invention, comprising extracted and purified RM, prepared in accordance with the present invention, may also be used in combination with synthetic high intensity sweeteners such as sucralose, potassium acesulfame, aspartame, alitame, saccharin, neohesperidin dihydrochalcone, cyclamate, neotame, dulcin, suosan advantame, salts thereof, and the like. Further, composition of the invention, comprising extracted and purified RM, prepared in accordance with the present invention, can be used in combination with natural sweetener suppressors such as gymnemic acid, hodulcin, ziziphin, lactisole, and others. Composition of the invention, comprising extracted and purified RM, prepared in accordance with the present invention, may also be combined with various umami taste enhancers. Composition of the invention, comprising extracted and purified RM, prepared in accordance with the present invention, can be mixed with umami tasting and sweet amino acids such as glutamate, aspartic acid, glycine, alanine, threonine, proline, serine, glutamate, lysine and tryptophan.

Composition of the invention, comprising extracted and purified RM, prepared in accordance with the present invention, can be used in combination with one or more additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof.

Composition of the invention, comprising extracted and purified RM, prepared in accordance with the present invention, may be combined with polyols or sugar alcohols. The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentanol, hexanol, heptanol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced which do not adversely affect the taste of the sweetener composition.

Composition of the invention, comprising extracted and purified RM, prepared in accordance with the present invention, may be combined with reduced calorie sweeteners such as D-tagatose, allulose, allose, L-sugars, L-sorbose, L-arabinose, and others.

Composition of the invention, comprising extracted and purified RM, prepared in accordance with the present invention, may also be combined with various carbohydrates. The term “carbohydrate” generally refers to aldehyde or ketone compounds substituted with multiple hydroxyl groups, of the general formula (CH2O)n, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfonyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, Psicose, turanose, allulose, allose, D-tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

Composition of the invention, comprising extracted and purified RM, prepared in accordance with the present invention, can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols); polyols; prebiotics, probiotics; phytoestrogens; soy protein; sulfides/thiols; amino acids; proteins; vitamins; and minerals. Functional ingredients also may be classified based on their health benefits, such as cardiovascular, cholesterol-reducing, and anti-inflammatory. Exemplary functional ingredients are provided in WO2013/096420, the contents of which is hereby incorporated by reference.

Composition of the invention, comprising extracted and purified RM, prepared in accordance with the present invention, may additionally comprise a secondary sweetening component. The secondary sweetening component is preferably selected from the group consisting of sucrose, erythritol, fructose, glucose, maltose, lactose, corn syrup (preferably high fructose), xylitol, sorbitol, or other sugar alcohols, inulin, miraculin, monetin, thaumatin and combinations thereof, and also non-natural sweeteners such as aspartame, neotame, saccharin, sucralose and combinations thereof. The natural sweetener compositions may be used alone or in combination with other secondary sweeteners, as described herein, and/or with one or more organic and amino acids, flavours and/or coloring agents.

Preferably the composition of the invention, comprising extracted and purified RM, prepared in accordance with the present invention, may be blended with one or more natural sweeteners, preferably crystalline fructose; and optionally with one or more flavouring agents, preferably thaumatin.

Preferably the composition of the invention, comprising extracted and purified RM, prepared in accordance with the present invention, may be blended with

A: One or more Stevia extracts selected from the group consisting of: RA (20, 40, 60, 80, 97, 97, 98, 99); STV (20, 40, 60, 80, 95, 97, 98, 99); RC (20, 40, 85, 90, 95, 97, 98, 99); RB (95/97); RD (95/97), and steviolbioside (95/97); and
B: None, one or more than one natural sweeteners selected from the group consisting of: high fructose syrup, crystalline fructose, sugar, isomaltulose, lactulose, soybean oligosaccharide, fructooligosaacharide, lactosucrose, xylooligosaacharide, erythritol, xylitol, sorbitol, mannitol, maltitol, lactitol, isomaltitol, and glycyrrhizin; and
C: None, one or more than one flavors selected from the group consisting of: thaumatin, monellin, miraculin, glycine, amino acids, L-glutamic acid, and fragrances.

Composition of the invention, comprising extracted and purified RM, prepared in accordance with the present invention, may be further processed using known methods to modify particle size and physical form. Methods such as agglomeration, spray-drying, drum drying and other forms of physical processing may be applied to adjust particle size in order to deliver better flow, hydration, or dissolution properties. The compositions may be provided in liquid forms, optionally containing one or more preservatives and/or processing aids, for ease-of-use in specific applications. Composition of the invention, comprising extracted and purified RM, prepared in accordance with the present invention may be co-processed with bulking agents such as maltodextrins and similar compounds to deliver products with controlled sweetness, dosing, potency, and handling properties.

Composition of the invention, comprising extracted and purified RM, prepared in accordance with the present invention, may be used in the preparation of various food products, beverages, medicinal formulations, chemical industrial products, among others. Exemplary applications/uses for the sweetener compositions include, but are not limited to: (a) food products, including canned food, preserved fruits, pre-prepared foods, soups, (b) beverages, including coffee, cocoa, juice, carbonated drinks, sour milk beverages, yogurt beverages, meal replacement beverages, and alcoholic drinks, such as brandy, whisky, vodka and wine; (c) grain-based goods—for example, bread and pastas, cookies, pastries, whether these goods are cooked, baked or otherwise processed; (d) fat-based products—such as margarines, spreads (dairy and non-dairy), peanut butter, peanut spreads, and mayonnaise; (d) Confectioneries—such as chocolate, candies, toffee, chewing gum, desserts, non-dairy toppings (for example Cool Whip®), sorbets, dairy and non-dairy shakes, icings and other fillings, (e) drug and medicinal formulations, particularly in coatings and flavourings; (f) cosmetics and health applications, such as for sweetening toothpaste; and (g) seasonings for various food products, such as soy sauce, soy sauce powder, soy paste, soy paste powder, catsup, marinade, steak sauce, dressings, mayonnaise, vinegar, powdered vinegar, frozen-desserts, meat products, fish-meat products, potato salad, bottled and canned foods, fruit and vegetables.

Composition of the invention, comprising extracted and purified RM, prepared in accordance with the present invention, may be formulated into premixes and sachets. Such premixes may then be added to a wide variety of foods, beverages and nutraceuticals. The purified natural sweetener compositions may, in one preferred form, be table top sweeteners.

While the forms of processes and compositions described herein constitute preferred embodiments of this invention, it is to be understood that the invention is not limited to these precise forms. As will be apparent to those skilled in the art, the various embodiments described above can be combined to provide further embodiments. Aspects of the present composition, method and process (including specific components thereof) can be modified, if necessary, to best employ the systems, methods, nodes and components and concepts of the invention. These aspects are considered fully within the scope of the invention as claimed. For example, the various methods described above may omit some acts, include other acts, and/or execute acts in a different order than set out in the illustrated embodiments.

Further, in the methods taught herein, the various acts may be performed in a different order than that illustrated and described. These and other changes can be made to the present systems, methods and articles in light of the above description. In general, in the following claims, the terms used should not be construed to limit the invention to the specific embodiments disclosed in the specification and the claims, but should be construed to include all possible embodiments along with the full scope of equivalents to which such claims are entitled. Accordingly, the invention is not limited by the disclosure, but instead its scope is to be determined entirely by the following claims.

All publications, patents and patent applications mentioned in the specification are indicative of the level of those skilled in the art to which this invention pertains. All such publications, patents and patent applications are incorporated by reference herein for the purpose cited to the same extent as if each was specifically and individually indicated to be incorporated by reference herein.

The following example illustrates a preferred but non-limiting embodiment of the present invention.

### EXAMPLES

### Example 1 Preparation of Crude Mother Liquor/Primary Extract (i.e. Starting Material)

One kg of the stevia leaves known to have a high content of Rebaudioside A were steeped with 2 kg of room temperature water having a pH of 7.3 in an agitation centrifuge. The leaves were agitated for 0.5 hour. The sweet water was filtered, the filtrate collected and the process repeated for a total of 5 steep/separation cycles. The pH of the sweet water filtrate solution was adjusted to pH 8.0 with approximately 30 grams of calcium hydroxide. After a rest time of about 1 hour, 50 grams of FeCl3 was added to the sweet water filtrate solution to further adjust the pH to 7.0. The solution was filtered and the resulting filtrate had a transmittance of about 68±2% at 325 nm.

The sweet water solution was then subjected to ion exchange columns consisting of both anion resin and cation resins, and then adsorption resin beds to de-salt, de-color and concentrate the sweet water. Subsequently, the resin beds were rinsed with ethanol (70%) to isolate the steviol glycosides from the resin beds. A sweet water solution with at least 96% transmittance at 325 nm was concentrated and spray dried. The yield was 130 grams of powder with a content of steviol glycosides of about 88.2%. The powder contained 57.6% Rebaudioside A content and 24.7% Stevioside content. The precipitate-free liquid remaining above the solid wherein the “supernate” or “supernatant” is the crude mother liquor/primary extract (i.e. starting material) of the invention.

Following a similar process to that outlined about, stevia leaves known to have a high content of Stevioside yield a powder of 130 grams of powder with a content of steviol glycosides of about 89.0% was obtained. The mixture contained 24.3% Rebaudioside A content and 59.6% Stevioside content.

### Example 2 Preparation of Crude Mother Liquor/Primary Extract (i.e. Starting Material)

The powder containing 57.6% Rebaudioside A (RebA) content isolated by the process of Example 1 was mixed with 2.5 times volume (w/v) of 92.0% ethanol at a temperature between about 45-50° C. for 1 hour with slow agitation. The RebA solution was filtered and a precipitate containing Rebaudioside A was dried to a powder. The resulting RebA powder had 89.2% RebA content. The powder was then mixed with three volumes (w/v) of 92% of ethanol, and maintained at a temperature of 45-50° C. for about 50 minutes with slow agitation. The precipitate was separated from the solution by filtration and the resulting precipitate comprised crystals of about 95.0% RebA content. The crystals were dissolved at room temperature in deionized water. The solution was concentrated and spray dried. The final RebA extract the “precipitate”) had a purity of about 97.5%. The precipitate-free liquid remaining above the solid wherein the “supernate” or “supernatant” is the crude mother liquor/primary extract (i.e. starting material) of the invention.

### Example 3 Preparation of Crude Mother Liquor/Primary Extract (i.e. Starting Material)

The powder containing 59.6% Stevioside (STV) content isolated by the process of Example 1 was mixed with 3 times volume (w/v) of mixture of methanol and ethanol having a methanol:ethanol ratio of 4:1. The STV solution was mixed at a temperature of 45-50° C. for 1 hour with slow agitation. The STV solution was filtered and a precipitate containing Stevioside was dried to a powder. The resulting STV powder had 85% STV content. The powder was then mixed with 2 volumes (w/v) of 90% of ethanol, and maintained at a temperature of 45-50° C. for about 50 minutes with slow agitation. The precipitate was separated from the solution by filtration and the resulting precipitate comprised crystals of about 96.2% STV content. The crystals were dissolved at room temperature in deionized water. The solution was concentrated and spray dried. The final STV extract had a purity of about 97.5%. The precipitate-free liquid remaining above the solid wherein the “supernate” or “supernatant” is the crude mother liquor/primary extract (i.e. starting material) of the invention.

### Example 4: Measuring Reb M in the Mother Liquor and then Extraction/Purification of Reb M

A Steviol Glycoside mother liquor is tested for Reb M content by liquid chromatographic analysis which—with an ideal for the starting material of 2.3%. Prepare Steviol Glycoside mother into feed liquor with concentration of 25 g/L, take 1,500 L feed liquor and allow to flow through 500 L DA-201-H macroporous resin column at a rate of 2.5 L/min. When the feed liquor flows through the resin column, the resin column conducts selective adsorption according to the polarity of different Steviol Glycosides components of the feed liquor, with 5.5 PH of adsorption environment. After 10 hours, at the end of absorption, 1,500 L of 77% alcohol resolves the resin Steviol Glycosides adsorbed on the resin. Intercept the eluent by segments in 100 L, and test the content of Reb M in eluent by liquid chromatography analysis. It is found that Steviol Glycoside Reb M starts to be eluted from segment of 800 L, after which the content of major components of Steviol Glycosides in eluent are compared with the parameters of content in feed liquor as shown in the following table:

| | | | | Total |
|---|---|---|---|---|
| Chromatographic | | | | glycoside |
| analysis object | STV | RA | RM | content |
| Feed liquor | 21.43% | 14.38% | 2.3% | 53.11% |
| 800 L-900 L | 19.86% | 12.98% | 5.89% | 51.73% |
| 900 L-1000 L | 21.83% | 15.21% | 10.08% | 60.12% |
| 1000 L-1,100 L | 19.81% | 13.12% | 20.89% | 68.82% |
| 1,100 L-1,200 L | 21.35% | 14.25% | 28.27% | 75.87% |
| 1,200 L-1,300 L | 23.85% | 16.02% | 33.01% | 85.88% |
| 1,300 L-1,400 L | 26.95% | 18.35% | 28.95% | 87.25% |
| 1,400 L-1,500 L | 27.30% | 16.92% | 20.15% | 79.37% |
| 1,500 L-1,600 L | 27.25% | 16.89% | 14.95% | 71.09% |

The above table shows that, the RM content in eluent at 1,200 L-1,300 L is up 33.01%, the total glycosides content reaches 85.88%. In the eluent at 1,100 L to 1,300 L, the RM content is 25-35%. RM enrichment reaches the maximum at 1,200 L-1,300 L fraction.

The eluent intercepted at 1,100 L-1,200 L is concentrated at 75° C., and the concentrated solid content is controlled at about 45%. The concentrate obtained is dried to produce crude Reb M. As tested by liquid chromatography, the Reb M content in the crude Steviol Glycosides is 28.27%.

The eluent intercepted at 1,300 L-1,400 L is concentrated at 70° C., and the concentrated solid content is controlled at about 45%. The concentrate obtained is dried to produce crude Reb M. As tested by liquid chromatography, the Reb M content in the crude Steviol Glycosides is 28.95%.

The eluent intercepted at 1,400 L-1,500 L is concentrated at 60° C., and the concentrated solid content is controlled at about 45%. The concentrate obtained is dried to produce crude Steviol Glycoside Reb M. As tested by liquid chromatography, the Reb M content in the crude Steviol Glycosides is 20.15%.

The eluent intercepted at 1,500 L-1,600 L is concentrated at 80° C., and the concentrated solid content is controlled at about 47%. The concentrate obtained is dried to produce crude Steviol Glycoside Reb M. As tested by liquid chromatography, the Reb M content in the crude Steviol Glycosides is 14.95%.

### Example 5: Further Processing-Preparation of Purified Stevia Product has the Steviol Glycoside RM Content of 95.73%

10 kg Steviol Glycosides with Reb M content of 28% produced in Example 4 is dissolved by stirring at 65° C. in the mixed solvent containing 30 kg methanol with mass concentration of 92% and 86% isopropyl alcohol by the ratio of 3:2 ratio, the solution is rapidly cooled to room temperature in 25 minutes, stirred for 10-20 minutes at intervals of 4 hours and stood for 48 hours, then solid-liquid separation is conducted to the crystal mixture to produce crystals with Reb M content of more than 80%. And then by secondary crystallization, crystal content is above 95%. The crystal solids filtered off are dissolved in desalted water with solution concentration adjusted to 27%, and then concentrated to about 43%, and the solution is dried to produce 2.0 kg purified Steviol Glycosides, such purified stevia product has the Steviol Glycoside RM content of 95.73%.

### Example 6: Further Processing—Preparation of Purified Stevia Product has the Steviol Glycoside RM Content of 95.34%

10 kg crude Steviol Glycoside Reb M content of 28.95% produced in Example 4 are dissolved by stirring at 67° C. in the mixed solvent containing 32 kg methanol with mass concentration of 90% and 87% isopropyl alcohol by the ratio of 3:2 ratio, the solution is rapidly cooled to room temperature in 25 minutes, stirred for 10-20 minutes at intervals of 5 hours and stood for 60 hours, then solid-liquid separation is conducted to the crystal mixture to produce crystals with Reb M content of more than 85%. And then by secondary crystallization, crystal content is above 95%. The crystal solids filtered off are dissolved in desalted water with solution concentration adjusted to about 25%, and then concentrated to about 45%, and the solution is dried to produce 2.2 kg purified Steviol Glycosides, such purified stevia product has the Steviol Glycoside RM content of 95.34%;

### Example 7: Further Processing—Preparation of Purified Stevia Product has the Steviol Glycoside RM Content of 96.02%

10 kg Steviol Glycoside product with Reb M content of 20.15% are dissolved by stirring at 75° C. in the mixed solvent containing 35 kg methanol with concentration of 88% and 90% isopropyl alcohol, the solution is rapidly cooled to room temperature in 25 minutes, stirred for 10-20 minutes at intervals of 6 hours and stood for 72 hours, then solid-liquid separation is conducted by a plate-and-frame filter press to the crystal mixture to produce crystals with Reb M content of more than 80%. And then by secondary crystallization, crystal content is above 95%. The crystal solids filtered off are dissolved in desalted water with solution concentration adjusted to 23%, and then concentrated to about 47%, and the solution is dried to produce 1.3 kg purified Steviol Glycosides, such purified stevia product has the Steviol Glycoside RM content of 96.02%;

Steviol Glycosides mentioned above may be in powder or crystalline; drying may use the current drying method of this invention such as vacuum drying and spray drying.

The above examples are only the preferred ones of this invention, and are not intended to limit this invention, any modification, equivalent replacement or improvement, etc. of which within the spirit and principles of this invention should be included in the protection of this invention.

### Example 8 Characterization of the Flavor Profiles of Existing Reb M Standards and Reb M of the Invention

### Samples

Reb M 95, Reb C 85, Reb D 95, Reb A 97, Sucrose, New Blends RA97 and RM95, New Blends RA97, RM95 and RC85, New Blends RM95, RA97 and Sucrose, New Blends RM95, RA97 and Erythritol, Purified Water.

TABLE 2
Sample test results

| | | | | | | Sensory | |
|---|---|---|---|---|---|---|---|
| | | Powder | Powder | Content/ | Color of | sweetness of | Sweetness of |
| No | Ingredient | Color | smell | 100 ml | Solution | Solution | Solution |
| 1 | Reb M 95 | White | No | 0.045 g | Colorless | Tastes | 9° |
| | | | abnormal | | | mellow with | |
| | | | odor | | | thick | |
| | | | | | | mouthfeel, | |
| | | | | | | without | |
| | | | | | | abnormal | |
| | | | | | | odor, | |
| | | | | | | astringency | |
| | | | | | | or aftertaste; | |
| | | | | | | minimal | |
| | | | | | | difference | |
| | | | | | | from sucrose | |
| 2 | Reb C 85 | White | No | 0.2 g | Colorless | Tastes | 9° |
| | | | abnormal | | | cooling with | |
| | | | odor | | | astringency, | |
| | | | | | | aftertaste, | |
| | | | | | | and licorice; | |
| | | | | | | larger | |
| | | | | | | difference | |
| | | | | | | from sucrose | |
| 3 | Reb D 95 | | No | 0.045 g | Colorless | Tastes | 9° |
| | | | abnormal | | | mellow with | |
| | | | odor | | | thick | |
| | | | | | | mouthfeel, | |
| | | | | | | without | |
| | | | | | | abnormal | |
| | | | | | | odor, | |
| | | | | | | astringency | |
| | | | | | | or aftertaste; | |
| | | | | | | minimal | |
| | | | | | | difference | |
| | | | | | | from sucrose | |
| 4 | Reb A 97 | White | No | 0.032 g | Colorless | Tastes | 9° |
| | | | abnormal | | | cooling with | |
| | | | odor | | | astringency, | |
| | | | | | | aftertaste, | |
| | | | | | | and licorice; | |
| | | | | | | larger | |
| | | | | | | difference | |
| | | | | | | from sucrose | |
| 5 | Sucrose | White | No | 9 g | Colorless | Tastes | 9° |
| | | | abnormal | | | mellow with | |
| | | | odor | | | thick | |
| | | | | | | mouthfeel, | |
| | | | | | | without | |
| | | | | | | abnormal | |
| | | | | | | odor, | |
| | | | | | | astringency | |
| | | | | | | or aftertaste | |
| 6 | New | White | No | 0.0334 g | Colorless | Tastes | 9° |
| | Blends | | abnormal | | | mellow with | |
| | RA97 & | | odor | | | thick | |
| | RM95 | | | | | mouthfeel, | |
| | | | | | | without | |
| | | | | | | abnormal | |
| | | | | | | odor, | |
| | | | | | | astringency | |
| | | | | | | or aftertaste; | |
| | | | | | | minimal | |
| | | | | | | difference | |
| | | | | | | from sucrose | |
| 7 | New | White | No | 0.037 g | Colorless | Tastes | 9° |
| | Blends | | abnormal | | | mellow with | |
| | RA97 & | | odor | | | thick | |
| | RM95 & | | | | | mouthfeel, | |
| | RC85 | | | | | without | |
| | | | | | | abnormal | |
| | | | | | | odor, | |
| | | | | | | astringency | |
| | | | | | | or aftertaste; | |
| | | | | | | minimal | |
| | | | | | | difference | |
| | | | | | | from sucrose | |
| 8 | New | White | No | 1.5 g | Colorless | Tastes | 9° |
| | Blends | | abnormal | | | mellow with | |
| | RM95 & | | odor | | | thick | |
| | RA97 & | | | | | mouthfeel, | |
| | Sucrose | | | | | without | |
| | | | | | | abnormal | |
| | | | | | | odor, | |
| | | | | | | astringency | |
| | | | | | | or aftertaste; | |
| | | | | | | minimal | |
| | | | | | | difference | |
| | | | | | | from sucrose | |
| 9 | New | White | No | 2.73 g | Colorless | Tastes | 9° |
| | Blends | | abnormal | | | mellow with | |
| | RM95 & | | odor | | | thick | |
| | RA97 & | | | | | mouthfeel, | |
| | Erythritol | | | | | without | |
| | | | | | | abnormal | |
| | | | | | | odor, | |
| | | | | | | astringency | |
| | | | | | | or aftertaste; | |
| | | | | | | minimal | |
| | | | | | | difference | |
| | | | | | | from sucrose | |

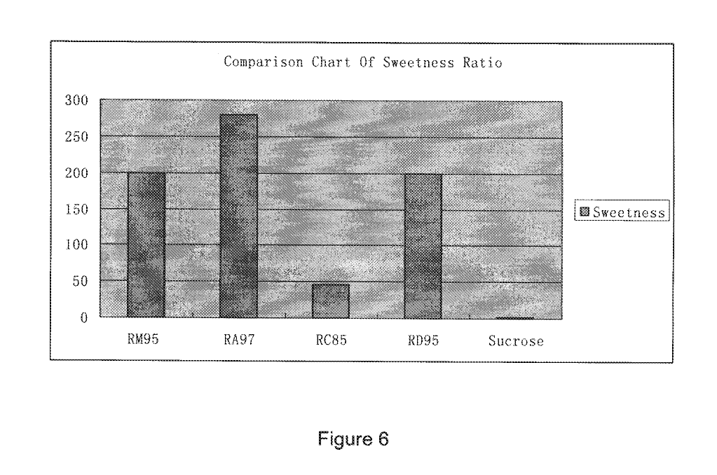

FIG. 6 is a bar graph showing the sweetness ratio of all the samples is tested as the concentration of 5% Sucrose solution, wherein RM95 is 200 times sweeter than sucrose, RA97 280 times, RC85 45 times, and RD95 200 times.

### Sensory Profiles for Sucrose and RM95

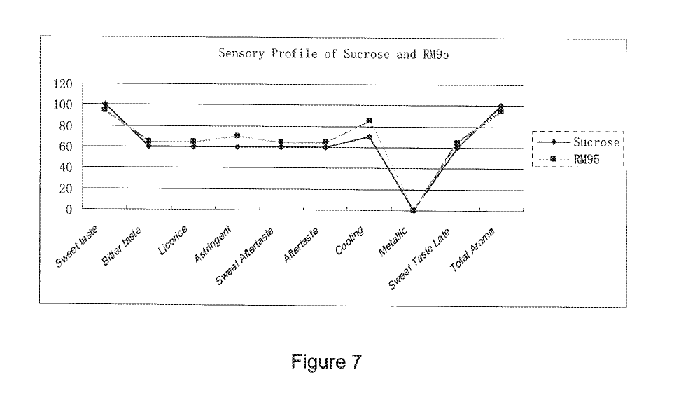

With reference to FIG. 7, there is direct of alignment of RM95 (extracted and purified n accordance with the process of the invention) and sugar. RM95 tasted mellow with thick mouthfeel, without abnormal odor, astringency, or aftertaste, having minimal difference with sucrose.

### Sensory Profile of RM95 and RA97

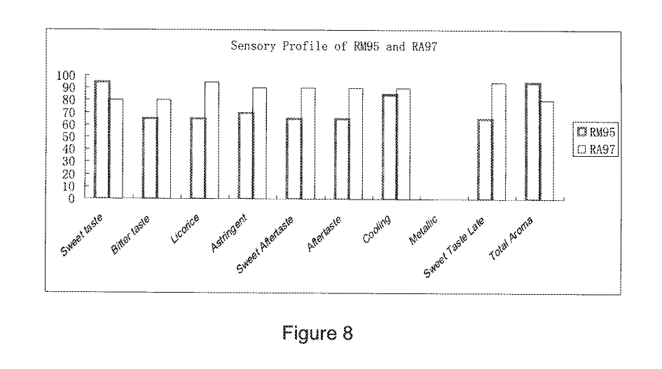

With reference to FIG. 8—RM95 and RA97 have a sizeable difference in the sensory area of licorice, astringency, sweet aftertaste, and sweet taste latency. RM95 tastes mellow with thick mouthfeel, without abnormal odor, astringency or aftertaste, having minimal difference with sucrose.

### Sensory Profiles for RM95, Sucrose and Other Steviol Glycosides

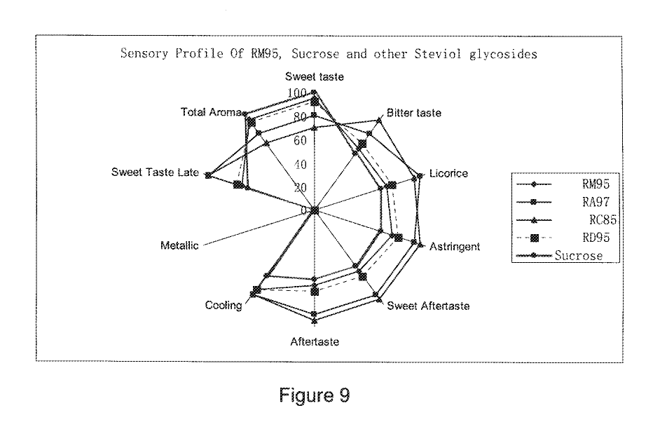

With reference to FIG. 9, RM95 tastes most nearly like sucrose. Sensory of RM95 is different from other Steviol glycosides such as RA97, RC85, but similar to RD95. Sensory of RM95 and RD95 are both most similar to sucrose.

### Time Intensity of RM95, Sucrose and Other Steviol Glycosides

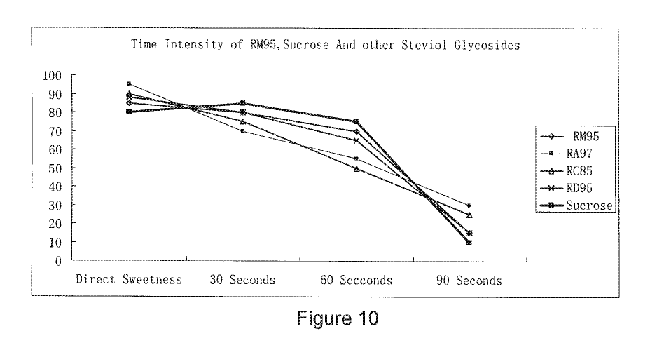

FIG. 10 shows RM95 having a duration of sweetness very similar to the profile of sugar.

### Sweet Releasing by Time

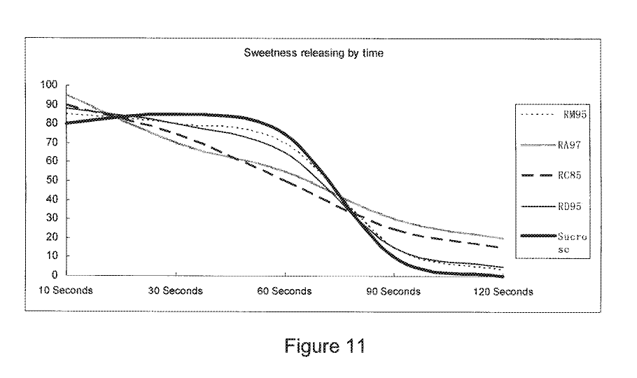

FIG. 11 shows, the sweetness of RM95 releases rapidly like sucrose; RA97, RC85 and RD95 all release slowly, and the sweetness taste lasts 120 seconds.

### Sensory Profiles for RM95, RA97, Sucrose and New Blends RA97&RM95

In FIG. 12, note that New Blend RA97&RM95 is named “M+A” for short. “M+A” is 268 times sweeter than sucrose, with the ratio of RM95 15% and RA97 85%.

### Sensory Profiles for RM95, RA97, RC85, Sucrose and New Blends RA97& RM95 & RC85

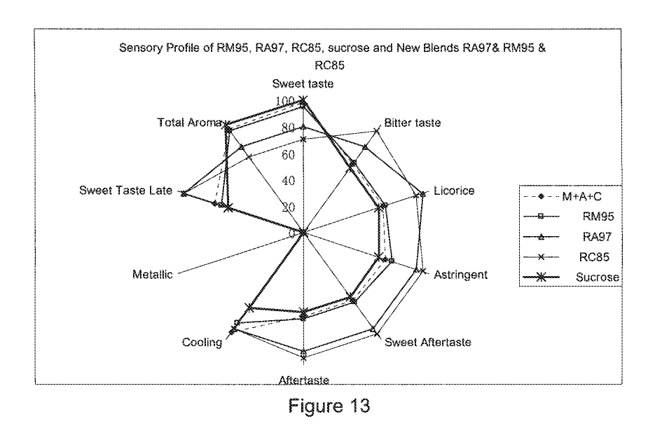

In FIG. 13, noted that New Blend RA97& RM95 & RC85 is named “M+A+C” for short. “M+A+C” is 241.4 times sweeter than sucrose, with the ratio of RM95 13%, RA97 75% and 12%.

### Sensory Profiles for RM95, RA97, Sucrose and New Blends RA97& RM95 & Sucrose

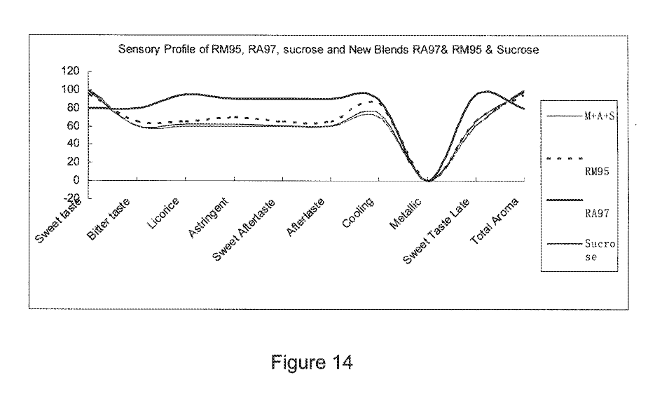

In FIG. 14, New Blend RA97& RM95 & Sucrose is named “M+A+S” for short. “M+A+S” is 6 times sweeter than sucrose, with the ratio of RM95 0.3%, RA97 1.6%, Sucrose 98.1%.

### Sensory Profiles for RM95, RA97, Sucrose and New Blends RA97& RM95 & Erythritol

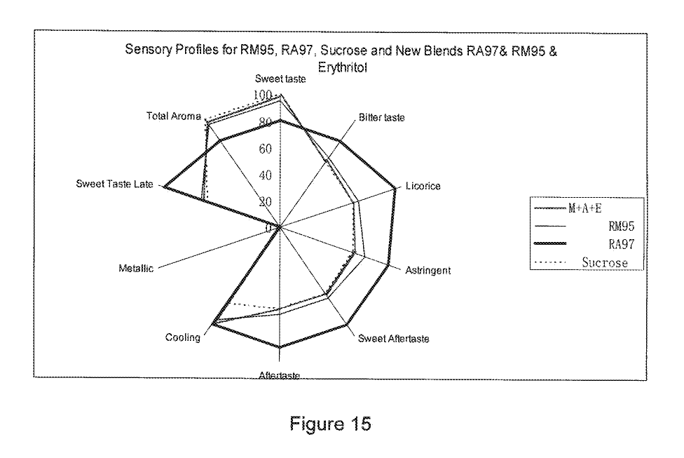

In FIG. 15, New Blend RA97& RM95 & erythritol is named “M+A+E” for short. “M+A+E” is 3.3 times sweeter than sucrose, with the ratio of RM95 0.15%, RA97 0.85%, Erythritol 99%.

All of the aforementioned data clearly shows the superior properties of RM (prepared in accordance with the process of the invention) over other steviol glycosides, across all metrics, as compared to sugar/sucrose.

1. A process for purifying Reb M from a crude mother liquor/Stevia rebaudiana primary extract, which comprises passing a solution of this primary extract through a multi-column system including a plurality of columns, in series, packed with a porous adsorbent resin to provide at least one column having adsorbed Reb M and eluting fractions with Reb M content from a final column in the series of columns, said last column having absorbed Reb M to provide an eluted solution with Reb M content.

2. The process of claim 1 wherein crude mother liquor/Stevia rebaudiana primary extract is a by-product of a prior extraction and purification process of i) Reb A; ii) STV; or ii) Reb A and STV.

3. The process of claim 1 wherein plurality is at least six columns.

4. The process of claim 1 wherein plurality is at least seven columns.

5. The process of claim 1 wherein plurality is eight columns.

6. The process of claim 1 which includes washing the multi-column system with a washing solution prior to eluting fraction with Reb M content in order to remove impurities.

7. The process of claim 1 which includes decolorizing the eluted solution.

8. The process of claim 1 which includes deionizing the eluted solution.

9. The process of claim 1 which includes further purifying the eluted solution by one or more of membrane filtration, ion exchange chromatography and activated carbon treatment.

10. A process for purifying Reb M from a crude mother liquor/Stevia rebaudiana primary extract comprises:
preparing a Stevia rebaudiana primary extract/mother liquor is in a form of dried powder, and wherein Reb M, measured in the dried powder is from about 2-3 g/L; and
passing the primary extract/mother liquor through a plurality of porous resin columns, in series, each of the columns packed with macroporous resin and eluting glycosides to provide an eluate from a final column (of said plurality of columns), said eluate comprising Reb M.

11. The process of claim 10 additionally comprising purifying the eluate by one of more of: decolorizing, evaporating, deionizing, concentrating, drying and crystallization.

12. The process of claim 10 comprising eight columns in series and a concentration of Reb M in the dried powder of between 2-3 g/L.

13. The process of claim 10 comprising 6-7 columns in series and a concentration of Reb M in the dried powder of over 3.0 g/L.

14. The process of claim 10 comprising 10 columns in series and a concentration of Reb M in the dried powder of less than 2.0 g/L.

15. A process for producing the crude mother liquor/Stevia rebaudiana primary extract for use in the process of claim 1, said process comprising the steps of:
drying Stevia leaves;
mixing and agitating the dried Stevia leaves with water to produce a water-leaves mixture;
filtering the water-leaves mixture to obtain an aqueous filtrate;
subjecting the aqueous filtrate to ion exchange columns and adsorption resin beds to isolate a steviol glycoside extract (A);
isolating a Stevioside (STV) extract from the steviol glycoside extract (A), leaving steviol glycoside extract (B);
isolating a Rebaudioside A extract from the steviol glycoside extract (B), leaving steviol glycoside extract (C); and
reserving steviol glycoside extract C, said steviol glycoside extract C being a crude mother liquor/primary extract.

16. The process of claim 15, wherein the mixture and agitation of the dried Stevia leaves with water is conducted with about 1 volume of water to about 15 volumes of water.

17. The process of claim 15, wherein the mixture and agitation of the dried Stevia leaves with water is conducted for about one hour to about five hours at about 5° C. to about 50° C.

18. The process of claim 15, wherein the water-leaves mixture is filtered to obtain an aqueous filtrate at room temperature using a compress filter for about three hours.

19. The process of claim 15, wherein the steviol glycoside extract is crystallized with an ethanol and methanol solvent to isolate and purify a Stevioside extract.

20. The process of claim 15 wherein, the steviol glycoside extract is crystallized with ethanol to isolate and purify Rebaudioside A extract.

21. A natural extract composition comprising Reb M, which is extracted and purified from Stevia rebaudiana according to the process of claim 1, wherein the composition is selected from any one of the group consisting of a natural extract, a natural sweetener, a food, a beverage and a nutraceutical.

22-25. (canceled)
